(19) United States
(12) Patent Application Publication
(10) Pub. No.: US 2023/0330265 A1
(43) Pub. Date: Oct. 19, 2023

(54) GENE THERAPY VECTOR FOR eEF1A2 AND USES THEREOF

(71) Applicant: UCL Business Ltd, London (GB)

(72) Inventors: Simon Nicholas WADDINGTON, London (GB); Rajvinder KARDA, Maidenhead (GB); Christopher Dean HERZOG, Fanwood, NJ (US); Joanna NG, London (GB); Chester SACRAMENTO, New York, NY (US); Stephanie SCHORGE, London (GB); David RICKS, New York, NY (US)

(21) Appl. No.: 18/017,502

(22) PCT Filed: Jul. 21, 2021
(86) PCT No.: PCT/EP2021/070455

§ 371 Date: Jan. 23, 2023

Related U.S. Application Data
(60) Provisional application No. 63/055,775, filed on Jul. 23, 2020.

(51) Int. Cl. A61K 48/00; C12N 15/86; C07K 14/47; C07K 14/61; A61P 25/28

(52) U.S. Cl. CPC A61K 48/0058; A61K 48/0041; A61P 25/28; C07K 14/47; C07K 14/61; C12N 15/86; C12N 2750/14143; C12N 2750/14171

(57) ABSTRACT

Provided herein is a gene therapy for neurological disease using a recombinant adeno-associated virus (rAAV) virion as a vector to express an eEF1A2 protein or functional variant thereof. The rAAV virion may use a neuron-specific promoter, e.g., a human synapsin 1 (hSYN) promoter. The capsid may be an AAV9 capsid or functional variant thereof. Other promoters or capsids may be used. Further provided are methods of treatment, such as by intracerebrally and/or intravenously of the rAAV virion, and other compositions and methods.

### CROSS REFERENCE TO RELATED APPLICATION

This application claims the benefit of priority to U.S. Provisional Pat. Application Serial No. 63/055,775 filed on Jul. 23, 2020, the contents of which are hereby incorporated by reference in their entireties.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ROPA_019_02WO_ST25.txt. The text file is about 101 kilobytes, created on Jul. 20, 2021, and is being submitted electronically via EFS-Web.

### BACKGROUND

The EEF1A2gene encodes Eukaryotic elongation factor 1, alpha-2 (eEF1A2), a protein involved in protein synthesis, suppression of apoptosis, and regulation of actin function and cytoskeletal structure. The mouse and human orthologs share identity at 462 of 463 amino acid positions. EEF1A2is a potential oncogene, as it is overexpressed in ovarian cancer. In research on ovarian cancer, a lentiviral vector encoding EEF1A2was used experimentally to transduce immortalized ovarian surface epithelial (IOSE) cells and thereby demonstrate that eEF1A2 promotes tumorigenesis in non-tumorigenic precursor cells. Sun et al. Int J Cancer. 123(8):1761-176 (2008).

EEF1A2is highly expressed in the central nervous system (CNS), as well as heart and muscle. Complete loss of EEF1A2in mice causes motor neuronal degeneration, a phenotype termed “wasted” whose genotype is termed wst. Davies et al. Sci Rep. 7:46019 (2017). Point mutations in the human EEF1A2gene have recently been demonstrated to variously cause epilepsy, intellectual disability, and/or autism. Cao et al. Human Molecular Genetics. 26(18):3545-3552 (2017); Lam et al. Mol Genet Genomic Med. 4(4):465-74 (2016); Nakajima et al. Clin Genet. 87(4):356-61 (2015).

Experiments using transgenic mice carrying wild-type EEF1A2on a bacterial artificial chromosome (BAC) have confirmed the wild-type Eefl1a2, when present during development, complements the wst genotype. Newbury et al. J. Bio. Chem. 282:2891-50 (2007).

EEF1A2-related disease is rare. Only about 100 individuals worldwide have been identified as having a mutation in EEF1A2. The etiology of disease remains poorly understood. Consequently, whether rescue of the disease phenotype by postnatal expression of wild-type EEF1A2could be achieved has been unclear. Furthermore, delivery of gene therapy to the CNS is challenging and unpredictable.

There is an unmet need for therapy for EEF1A2-related disease. The gene therapies provided herein address this need.

### SUMMARY

The present invention relates generally to gene therapy for neurological disease or disorders using adeno-associated virus (AAV)-based delivery of a polynucleotide encoding eEF1A2 or a functional variant thereof.

In one aspect, the disclosure provides a recombinant adeno-associated virus (rAAV) virion, comprising a capsid and a vector genome, wherein the vector genome comprises a polynucleotide sequence encoding an eEF1A2 protein or a functional variant thereof, operatively linked to a promoter. The promoter may be a neuron-specific promoter, e.g., a human synapsin 1 (hSYN) promoter. The capsid may be an AAV9 capsid or functional variant thereof. Other promoters or capsids may be used.

In another aspect, the disclosure provides a method of treating and/or preventing a neurological disease or disorder in a subject in need thereof, comprising administering the rAAV virion of the disclosure, or a pharmaceutical composition thereof, to the subject. The rAAV virion may be administered intracerebrally and/or intravenously.

In another aspect, the disclosure provides a method of expressing eEF1A2 in brain of a subject in need thereof, comprising administering the rAAV virion of the disclosure, or a pharmaceutical composition thereof, to the subject. The rAAV virion may be administered intracerebrally and/or intravenously.

In further aspects, the disclosure provides polynucleotides (e.g., vector genomes), pharmaceutical compositions, kits, and other compositions and methods.

Various other aspects and embodiments are disclosed in the detailed description that follows. The invention is limited solely by the appended claims.

### BRIEF DESCRIPTION OF FIGURES

FIG. 1 shows a domain diagram of eEF1A2 showing point mutations associated with disease.

FIG. 2 shows a vector diagram of a non-limiting example of a vector genome.

FIG. 3 shows a vector diagram of a non-limiting example of a vector genome.

FIG. 4 shows a vector diagram of a non-limiting example of a vector genome.

FIG. 5 shows a vector diagram of a non-limiting example of a vector genome.

FIG. 6 shows a vector diagram of a non-limiting example of a vector genome.

FIG. 7 shows immunofluorescence microscopy of mice after neonatal injection, intracerebrally (IC) or intravenously (IV), of AAV9-hSyn-eEF1A2-2A-eGFP or control. Scale bar, 300 µm.

FIG. 8A shows immunohistochemical analysis of mice after neonatal injection, intracerebrally (IC) or intravenously (IV), of AAV9-hSyn-eEF1A2-2A-eGFP or control. FIG. 8B shows a magnified view of the same slides. Scale bar, 300 µm.

FIG. 9A shows survival in untreated wst/wst (null) mice compared to intracerebrally (IC), intravenously (IV) or a combination of both (IC+IV) treated mice.

FIG. 9B shows weight loss in untreated wst/wst (null) mice compared to intracerebrally (IC), intravenously (IV) or a combination of both (IC+IV) treated mice.

FIG. 9C shows rotarod testing in untreated wst/wst (null) mice compared to intracerebrally (IC), intravenously (IV) or a combination of both (IC+IV) treated mice.

FIG. 9D shows inverted grid testing in untreated wst/wst (null) mice compared to intracerebrally (IC), intravenously (IV) or a combination of both (IC+IV) treated mice.

FIG. 9E shows eEF1A2 expression in untreated wst/wst (null) mice compared to intracerebrally (IC), intravenously (IV) or a combination of both (IC+IV) treated mice. Scale bar, 125 µm.

FIG. 9F shows eEF1A2 expression in untreated wst/wst (null) mice compared to intracerebrally (IC), intravenously (IV) or a combination of both (IC+IV) treated mice.

FIGS. 10A-10K shows comparisons of AAV9 vectors comprising the vector genomes shown in FIG. 2 (“V1”), FIG. 3 (“V2”), FIG. 4 (“V3”) and FIG. 6 (“V4”) administered at 2e10¹¹ vg/animal.

FIG. 10A shows a Kaplan-Meier survival plot of FBS treated wildtype, wst/wst, intracerebroventricular treated wst/wst animals with V1, V2,V3 and V4 gene therapy treated.

FIG. 10B shows weights of mice (Data means ± S.E.M.). Animals were weighed daily until postnatal age 35 and weekly thereafter until timed sacrifice point P60 or humane endpoint 15% weight loss.

FIG. 10C shows muscle strength assessment by inverted grid at day P15.

FIG. 10D shows muscle strength assessment by rota rod at day P15.

FIG. 10E shows muscle strength assessment by inverted grid at day P23.

FIG. 10F shows muscle strength assessment by rota rod at day P23.

FIG. 10G shows representative immunostaining for eEF1A2 throughout the brain by free floating immunohistochemistry with wildtype FBS (Formulation buffer solution) as physiological reference (n=4-5 per group, scale bar 250 µm).

FIG. 10H shows representative immunohistofluorescence in cortical brain regions for neurons (NeuN marker) co-expressing eEF1A2 (n=4.5 per group, 200 µm).

FIG. 10I shows representative immunoblot for eEF1A2 in brain with quantification showing eEF1A2 expression throughout the brain achieved with all gene therapy vectors with higher expression in midbrain, cerebellar and hindbrain regions compared to V4 vector (Data means ± S.E.M, two-way ANOVA).

FIG. 10J shows qPCR for human eEF1A2 transcript expression in forebrain showing highest mRNA expression with V1 vector (Data means ± S.E.M, two-way ANOVA).

FIG. 10K shows qPCR for human eEF1A2 cortex expression in cortex showing highest mRNA expression with V1 vector (Data means ± S.E.M, two-way ANOVA).

FIGS. 11A-11C

FIG. 11A shows Kaplan-Meier survival plot of FBS treated wildtype, FBS treated D252H/+, knock-outs (D252H-/-), intracerebroventricular V3 gene therapy treated D252H-/-(wildtype FBS n = 12, D252H/+ FBS n= 5, D252H-/- FBS n= 4 & 2 × 10¹¹ vg/pup, V3 treated n= 5).

FIG. 11B shows body weight across time (Data means ± S.E.M., one-way ANOVA, and Dunnett’s multiple comparison).

FIG. 11C shows motor assessment by rota rod (Data means ± S.E.M., two-way ANOVA, and Dunnett’s multiple comparison).

FIG. 12A shows Kaplan-Meier survival plot of Del22ex3 receiving V3 high dose (2× 10¹¹ vg/pup, n=5), Del22ex3 receiving V3 low dose (2 × 10¹⁰ vg/pup, n=5), Del22ex3 controls receiving formulation buffer solution (n=3) and wildtype controls receiving formulation buffer solution (n=6).

FIG. 12B shows body weight across time (Data means ± S.E.M.).

FIG. 12C shows motor assessment by grip strength manometry P22-25 (Data means ± S.E.M)

FIG. 12D shows grip strength manometry at P23 (Data means ± S.E.M., two-way ANOVA, and Tukey’s multiple comparison).

FIG. 12E shows motor assessment by rotarod P22-25 (Data means ± S.E.M)

FIG. 12F shows rotarod data at P24 (Data means ± S.E.M., two-way ANOVA, and Tukey’s multiple comparison).

FIG. 12G shows Neurological scores from P21-25.

### DETAILED DESCRIPTION OF THE INVENTION

### Definitions

The section headings are for organizational purposes only and are not to be construed as limiting the subject matter described to particular aspects or embodiments.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus a query sequence “shares at least x% identity to” a subject sequence if, when the query sequence is aligned to the subject sequence, at least x% (rounded down) of the residues in the subject sequence are aligned as an exact match to a corresponding residue in the query sequence. Where the subject sequence has variable positions (e.g., residues denoted X), an alignment to any residue in the query sequence is counted as a match.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operable linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with central nervous system. A subject may have a mutation in an EEF1A2 gene or deletion of all or a part of EEF1A2 gene, or of gene regulatory sequences, that causes aberrant expression of the eEF1A2 protein. “Subject” and “patient” are used interchangeably herein. The subject treated by the methods described herein may be an adult or a child. Subjects may range in age.

As used herein, the term “variant” or “functional variant” refer, interchangeably, to a protein that has one or more amino-acid substitutions, insertions, or deletion compared to a parental protein that retains one or more desired activities of the parental protein.

As used herein, “genetic disruption” refers to a partial or complete loss of function or aberrant activity in a gene. For example, a subject may suffer from a genetic disruption in expression or function in the EEF1A2 gene that decreases expression or results in loss or aberrant function of the eEF1A2 protein in at least some cells (e.g., neurons) of the subject.

As used herein, “treating” refers to ameliorating one or more symptoms of a disease or disorder. The term “preventing” refers to delaying or interrupting the onset of one or more symptoms of a disease or disorder or slowing the progression of eEF1A2 related neurological disease or disorder.

### Eef1a2 Protein or Polynucleotide

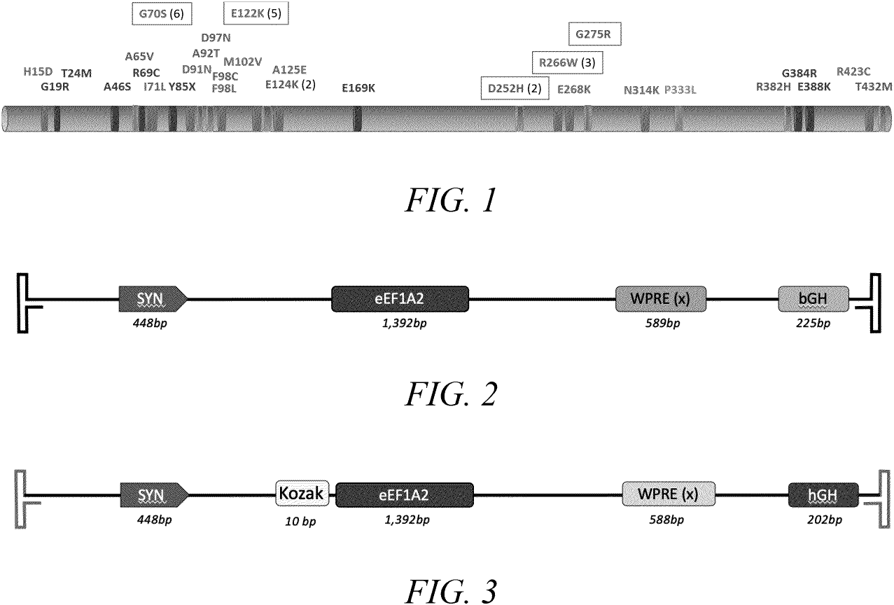

The present disclosure contemplates compositions and methods of use related to Elongation factor 1-alpha 2 (eEF1A2) protein. Various mutations in EEF1A2, illustrated in FIG. 1, are known to be associated with neurological disorders, including epilepsy, intellectual disability, and/or autism. Both inherited and de novo mutations have been observed. In some cases, a heterozygous missense mutation is sufficient to cause disease.

The polypeptide sequence of eEF1A2 is as follows:

MGKEKTHINIVVIGHVDSGKSTTTGHLIYKCGGIDKRTIEKFEKEAAEMG
KGSFKYAWVLDKLKAERERGITIDISLWKFETTKYYITIIDAPGHRDFIK
NMITGTSQADCAVLIVAAGVGEFEAGISKNGQTREHALLAYTLGVKQLIV
GVNKMDSTEPAYSEKRYDEIVKEVSAYIKKIGYNPATVPFVPISGWHGDN
MLEPSPNMPWFKGWKVERKEGNASGVSLLEALDTILPPTRPTDKPLRLPL
QDVYKIGGIGTVPVGRVETGILRPGMVVTFAPVNITTEVKSVEMHHEALS
EALPGDNVGFNVKNVSVKDIRRGNVCGDSKSDPPQEAAQFTSQVIILNHP
GQISAGYSPVIDCHTAHIACKFAELKEKIDRRSGKKLEDNPKSLKSGDAA
IVEMVPGKPMCVESFSQYPPLGRFAVRDMRQTVAVGVIKNVEKKSGGAGK
VTKSAQKAQKAGK(SEQ ID NO: 1).

In some embodiments, the eEF1A2 protein comprises a polypeptide sequence at least 75%, 80%, 85%, 90%, 92%, 93%, 94%, 95%, 96%, 97%, 98%, 99%, or 100% identical to SEQ ID NO: 1).

In some embodiments, the disclosure provides a recombinant adeno-associated virus (rAAV) virion, comprising a capsid and a vector genome, wherein the vector genome comprises a polynucleotide sequence encoding the eEF1A2 protein or a functional variant thereof, operatively linked to a promoter. In some embodiments, the disclosure provides a recombinant adeno-associated virus (rAAV) virion, comprising a capsid and a vector genome, wherein the vector genome comprises a polynucleotide sequence encoding an eEF1A2 protein, operatively linked to a promoter. The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGCAAGGAGAAGACCCACATCAACATCGTGGTCATCGGCCACGTGGA
CTCCGGAAAGTCCACCACCACGGGCCACCTCATCTACAAATGCGGAGGTA
TTGACAAAAGGACCATTGAGAAGTTCGAGAAGGAGGCGGCTGAGATGGGG
AAGGGATCCTTCAAGTATGCCTGGGTGCTGGACAAGCTGAAGGCGGAGCG
TGAGCGCGGCATCACCATCGACATCTCCCTCTGGAAGTTCGAGACCACCA
AGTACTACATCACCATCATCGATGCCCCCGGCCACCGCGACTTCATCAAG
AACATGATCACGGGTACATCCCAGGCGGACTGCGCAGTGCTGATCGTGGC
GGCGGGCGTGGGCGAGTTCGAGGCGGGCATCTCCAAGAATGGGCAGACGC
GGGAGCATGCCCTGCTGGCCTACACGCTGGGTGTGAAGCAGCTCATCGTG
GGCGTGAACAAAATGGACTCCACAGAGCCGGCCTACAGCGAGAAGCGCTA
CGACGAGATCGTCAAGGAAGTCAGCGCCTACATCAAGAAGATCGGCTACA
ACCCGGCCACCGTGCCCTTTGTGCCCATCTCCGGCTGGCACGGTGACAAC
ATGCTGGAGCCCTCCCCCAACATGCCGTGGTTCAAGGGCTGGAAGGTGGA
GCGTAAGGAGGGCAACGCAAGCGGCGTGTCCCTGCTGGAGGCCCTGGACA
CCATCCTGCCCCCCACGCGCCCCACGGACAAGCCCCTGCGCCTGCCGCTG
CAGGACGTGTACAAGATTGGCGGCATTGGCACGGTGCCCGTGGGCCGGGT
GGAGACCGGCATCCTGCGGCCGGGCATGGTGGTGACCTTTGCGCCAGTGA
ACATCACCACTGAGGTGAAGTCAGTGGAGATGCACCACGAGGCTCTGAGC
GAAGCTCTGCCCGGCGACAACGTCGGCTTCAATGTGAAGAACGTGTCGGT
GAAGGACATCCGGCGGGGCAACGTGTGTGGGGACAGCAAGTCTGACCCGC
CGCAGGAGGCTGCTCAGTTCACCTCCCAGGTCATCATCCTGAACCACCCG
GGGCAGATTAGCGCCGGCTACTCCCCGGTCATCGACTGCCACACAGCCCA
CATCGCCTGCAAGTTTGCGGAGCTGAAGGAGAAGATTGACCGGCGCTCTG
GCAAGAAGCTGGAGGACAACCCCAAGTCCCTGAAGTCTGGAGACGCGGCC
ATCGTGGAGATGGTGCCGGGAAAGCCCATGTGTGTGGAGAGCTTCTCCCA
GTACCCGCCTCTCGGCCGCTTCGCCGTGCGCGACATGAGGCAGACGGTGG
CCGTAGGCGTCATCAAGAACGTGGAGAAGAAGAGCGGCGGCGCCGGCAAG
GTCACCAAGTCGGCGCAGAAGGCGCAGAAGGCGGGCAAG(SEQ ID NO:
2).

The polynucleotide sequence encoding the eEF1A2 protein may be codon optimized.

The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGTAAAGAAAAAACACATATTAATATAGTAGTAATCGGTCATGTTGA
CTCTGGAAAATCTACTACTACAGGACATTTGATTTATAAATGTGGAGGAA
TTGATAAAAGAACAATAGAAAAATTTGAAAAAGAAGCTGCTGAAATGGGT
AAAGGTAGTTTTAAATATGCTTGGGTTTTGGATAAATTGAAAGCTGAAAG
AGAAAGAGGAATTACAATTGATATTTCTTTGTGGAAATTTGAAACTACAA
AATATTATATAACAATAATAGATGCTCCTGGACATAGAGATTTTATTAAA
AATATGATTACAGGAACTTCTCAAGCAGATTGTGCTGTTTTGATAGTAGC
AGCAGGAGTTGGTGAATTCGAAGCAGGCATTTCTAAAAATGGACAAACTA
GAGAACATGCTTTGTTGGCTTATACATTGGGCGTAAAACAATTGATTGTA
GGAGTTAATAAAATGGATTCTACTGAACCTGCATATTCTGAAAAAAGATA
TGATGAAATAGTAAAAGAAGTTTCTGCTTATATTAAAAAAATTGGTTATA
ATCCTGCTACAGTTCCATTTGTTCCTATTTCTGGATGGCATGGAGATAAT
ATGTTGGAACCTAGTCCTAATATGCCTTGGTTTAAAGGATGGAAAGTTGA
AAGGAAAGAAGGAAATGCATCAGGAGTCTCCTTGTTGGAAGCTTTGGATA
CAATCTTGCCTCCAACAAGACCTACAGATAAACCTTTGAGATTGCCTCTT
CAAGATGTATATAAAATAGGAGGAATAGGAACAGTGCCAGTTGGAAGAGT
AGAAACAGGTATATTGAGACCTGGAATGGTTGTAACATTTGCACCAGTTA
ATATAACTACTGAAGTAAAATCTGTTGAAATGCATCATGAAGCTTTGTCT
GAAGCTCTTCCTGGAGATAATGTAGGATTTAATGTTAAAAATGTAAGTGT
AAAAGATATAAGAAGAGGAAATGTATGTGGTGATAGTAAATCAGATCCAC
CTCAAGAAGCAGCTCAATTTACATCACAAGTAATAATATTGAATCATCCT
GGACAAATTTCTGCAGGATATTCACCAGTAATAGATTGTCATACAGCACA
TATAGCTTGTAAATTTGCTGAATTGAAAGAAAAAATTGATAGAAGAAGTG
GAAAAAAACTTGAAGATAATCCTAAATCATTGAAATCAGGAGATGCAGCT
ATTGTAGAAATGGTACCTGGAAAACCAATGTGTGTAGAATCTTTTTCTCA
ATATCCACCTCTCGGAAGATTTGCTGTTAGAGATATGAGACAAACAGTTG
CAGTAGGAGTTATTAAAAATGTAGAAAAAAAAAGCGGAGGTGCAGGAAAG
GTTACAAAATCCGCACAAAAAGCTCAAAAAGCTGGTAAATAA(SEQ ID
NO: 4).

The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGCAAAGAAAAAACACATATAAACATTGTCGTTATCGGACACGTTGA
TTCTGGTAAAAGTACAACAACCGGTCACTTGATATACAAATGCGGGGGTA
TAGACAAACGCACTATTGAAAAGTTCGAGAAAGAAGCTGCGGAGATGGGC
AAAGGCTCATTCAAGTACGCGTGGGTACTCGATAAGTTGAAAGCTGAACG
CGAGAGGGGAATCACCATAGACATCTCACTTTGGAAATTCGAGACAACCA
AGTATTACATAACTATTATAGATGCCCCAGGCCACAGGGATTTCATTAAA
AATATGATAACCGGCACATCTCAAGCCGATTGCGCCGTACTCATCGTCGC
CGCTGGTGTGGGTGAGTTCGAGGCAGGTATTTCTAAAAATGGCCAGACAC
GCGAACATGCTCTTCTGGCTTATACACTCGGGGTTAAACAGCTCATAGTA
GGAGTGAATAAGATGGACTCCACTGAACCCGCCTATTCAGAGAAGCGCTA
TGACGAAATTGTAAAGGAGGTCTCAGCATATATTAAAAAAATTGGCTATA
ACCCAGCCACGGTGCCATTCGTCCCGATTAGTGGATGGCATGGTGACAAT
ATGCTGGAACCAAGTCCCAATATGCCTTGGTTTAAGGGTTGGAAAGTAGA
GCGGAAAGAGGGTAATGCTTCCGGCGTGTCATTGCTGGAGGCGCTTGACA
CGATACTCCCACCCACAAGGCCAACTGATAAGCCACTCCGATTGCCCTTG
CAGGACGTGTACAAGATTGGGGGAATTGGGACTGTGCCCGTCGGGCGCGT
GGAGACGGGCATCCTCAGACCTGGGATGGTAGTCACTTTTGCCCCCGTCA
ACATAACGACTGAAGTTAAATCAGTGGAAATGCATCACGAAGCTTTGAGT
GAGGCGCTTCCCGGAGATAACGTTGGATTTAATGTCAAAAATGTCTCCGT
TAAAGATATAAGAAGAGGAAACGTCTGCGGTGACTCAAAGTCAGACCCAC
CACAGGAGGCTGCTCAATTTACGAGTCAAGTAATAATTCTGAATCACCCT
GGGCAAATAAGTGCGGGATACTCTCCAGTCATCGATTGTCACACCGCCCA
TATTGCATGTAAGTTCGCAGAACTTAAGGAAAAGATCGACCGAAGAAGCG
GAAAAAAATTGGAAGATAATCCGAAAAGTTTGAAAAGCGGTGACGCGGCG
ATTGTAGAGATGGTCCCTGGCAAACCGATGTGTGTGGAGTCTTTCAGTCA
ATATCCACCACTCGGTCGCTTTGCCGTGCGGGATATGCGACAGACCGTTG
CTGTCGGCGTAATAAAAAACGTCGAAAAAAAGAGCGGTGGGGCTGGAAAA
GTTACAAAATCCGCTCAAAAGGCACAGAAGGCGGGCAAGTGA(SEQ ID
NO: 5).

The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGTAAAGAAAAGACCCACATTAACATAGTAGTAATCGGTCATGTTGA
CTCTGGGAAAAGCACTACTACCGGACATTTGATCTATAAATGTGGGGGCA
TCGACAAAAGAACGATAGAGAAGTTTGAGAAGGAGGCGGCGGAGATGGGT
AAAGGTAGTTTTAAGTACGCTTGGGTTTTGGACAAATTGAAAGCCGAGCG
CGAGCGCGGCATTACCATTGACATTTCTCTCTGGAAATTCGAAACTACGA
AGTATTATATAACAATAATAGACGCCCCCGGCCATCGGGACTTTATTAAA
AACATGATTACAGGAACTAGCCAAGCAGATTGTGCTGTGCTGATAGTAGC
GGCAGGGGTCGGGGAGTTCGAAGCAGGCATCTCTAAAAATGGACAAACTC
GAGAGCACGCCTTGTTGGCTTATACCTTGGGCGTAAAGCAGCTGATCGTA
GGAGTTAATAAAATGGATTCCACTGAACCCGCATATAGCGAAAAGCGATA
TGACGAAATAGTAAAGGAAGTCTCAGCTTATATCAAGAAAATCGGTTACA
ATCCTGCGACGGTTCCATTCGTTCCTATCTCCGGGTGGCACGGCGATAAT
ATGCTTGAGCCCAGTCCCAATATGCCCTGGTTCAAGGGGTGGAAGGTTGA
GAGGAAGGAAGGCAATGCATCAGGCGTCAGCTTGTTGGAAGCTCTCGACA
CCATCCTGCCGCCCACGAGGCCCACAGACAAACCGTTGCGACTGCCTCTT
CAAGATGTATACAAAATAGGCGGGATAGGAACCGTGCCGGTTGGACGAGT
AGAGACGGGTATACTGCGGCCCGGAATGGTCGTGACGTTTGCACCCGTGA
ATATAACTACTGAGGTGAAGAGCGTCGAGATGCACCATGAAGCGCTGAGT
GAAGCTCTCCCTGGCGATAACGTAGGGTTCAACGTGAAAAACGTAAGTGT
AAAGGATATAAGGCGCGGAAATGTATGTGGTGACAGTAAAAGCGACCCGC
CGCAAGAGGCGGCGCAATTCACATCACAGGTAATAATATTGAATCACCCC
GGCCAAATTTCCGCAGGCTACTCACCAGTCATAGATTGCCACACCGCCCA
CATAGCTTGTAAGTTCGCTGAGTTGAAAGAGAAGATTGATAGACGAAGTG
GGAAGAAACTTGAAGACAATCCGAAGTCCCTGAAGTCCGGTGACGCAGCG
ATTGTAGAAATGGTACCGGGCAAGCCAATGTGTGTAGAGTCTTTCAGCCA
GTACCCACCACTGGGGCGGTTCGCGGTGCGAGACATGAGGCAAACGGTTG
CGGTCGGCGTCATTAAAAATGTCGAAAAAAAGAGTGGCGGTGCAGGTAAG
GTCACAAAAAGCGCACAAAAGGCCCAGAAAGCCGGTAAGTGA(SEQ ID
NO: 6).

The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGAAAGGAAAAAACTCACATAAACATTGTCGTCATCGGTCACGTAGA
CAGTGGCAAATCAACGACCACTGGACATCTCATCTATAAGTGTGGCGGTA
TTGACAAACGCACTATCGAGAAATTCGAAAAGGAGGCTGCTGAGATGGGC
AAAGGCTCTTTCAAGTACGCATGGGTCCTGGATAAGCTGAAAGCGGAGCG
AGAGAGAGGGATCACCATCGATATATCTCTGTGGAAATTTGAAACCACCA
AGTACTACATCACAATTATTGATGCCCCAGGTCATAGGGATTTTATCAAG
AACATGATCACCGGGACAAGCCAAGCCGACTGCGCAGTTCTCATAGTGGC
GGCTGGAGTAGGGGAGTTTGAAGCAGGGATATCTAAGAATGGACAGACCC
GCGAGCACGCCTTGCTGGCCTACACCCTGGGAGTGAAGCAGCTCATAGTT
GGCGTCAATAAGATGGACAGCACCGAACCCGCCTACAGTGAGAAGAGGTA
TGACGAGATTGTGAAGGAGGTTTCTGCTTACATTAAAAAGATTGGCTATA
ACCCAGCTACTGTCCCATTCGTTCCAATCAGCGGCTGGCACGGTGATAAC
ATGCTGGAGCCTAGTCCCAACATGCCGTGGTTCAAGGGGTGGAAGGTTGA
ACGCAAGGAGGGGAATGCCTCAGGCGTTTCCCTGCTGGAGGCCCTCGATA
CAATACTCCCCCCGACCCGGCCTACAGATAAACCGCTGCGACTGCCTCTT
CAGGACGTGTATAAAATCGGGGGAATCGGCACAGTGCCCGTGGGCAGGGT
AGAGACTGGCATCTTGCGGCCTGGAATGGTAGTCACCTTTGCCCCGGTTA
ATATCACAACGGAGGTGAAATCTGTGGAGATGCATCACGAAGCACTGAGC
GAGGCTCTGCCTGGTGACAACGTGGGATTTAACGTCAAAAACGTGTCAGT
CAAGGACATCCGCCGCGGTAACGTTTGCGGAGATTCTAAGTCCGATCCCC
CCCAGGAGGCAGCCCAATTTACCTCCCAAGTGATCATTCTGAATCACCCA
GGCCAAATTTCCGCCGGGTATTCCCCTGTGATTGACTGTCACACAGCACA
CATCGCATGCAAATTCGCCGAACTCAAGGAGAAAATTGATCGGAGAAGCG
GTAAAAAACTGGAGGACAACCCAAAGTCCCTCAAGTCTGGGGATGCCGCC
ATCGTGGAGATGGTACCAGGCAAACCTATGTGCGTGGAAAGTTTTAGCCA
GTACCCTCCACTGGGTCGCTTTGCTGTTCGGGATATGCGGCAGACAGTAG
CGGTTGGGGTCATAAAAAACGTCGAGAAAAAGAGCGGAGGAGCTGGGAAA
GTTACCAAATCCGCACAGAAGGCACAAAAAGCCGGAAAATGA(SEQ ID
NO: 7).

The polynucleotide encoding the eEF1A2 protein may comprise a polynucleotide sequence at least 75%, 80%, 85%, 90%, 92%, 93%, 94%, 95%, 96%, 97%, 98%, 99%, or 100% identical to:

ATGGGCAAAGAGAAAACACATATTAACATTGTTGTTATCGGGCACGTTGA
TAGCGGCAAGTCCACTACCACTGGCCATCTGATTTACAAGTGCGGCGGAA
TCGATAAACGAACTATTGAAAAGTTCGAAAAAGAAGCCGCCGAGATGGGA
AAGGGCTCCTTTAAATACGCTTGGGTCCTCGATAAACTCAAAGCAGAACG
GGAGAGAGGAATCACCATCGATATATCCTTGTGGAAGTTCGAAACTACAA
AATATTACATTACCATCATTGATGCGCCTGGGCACCGCGACTTCATTAAG
AACATGATTACTGGCACCTCTCAAGCCGACTGCGCAGTGTTGATCGTAGC
CGCAGGCGTCGGGGAGTTCGAAGCTGGGATCAGCAAGAACGGGCAGACTA
GGGAACACGCTCTGCTCGCATATACTCTTGGCGTGAAACAGTTGATCGTT
GGCGTGAACAAGATGGATTCAACTGAGCCTGCCTATTCTGAGAAACGATA
CGACGAGATTGTGAAAGAGGTTTCAGCTTACATCAAGAAAATTGGGTATA
ATCCCGCAACAGTTCCCTTCGTGCCCATCTCTGGGTGGCACGGCGACAAC
ATGCTCGAACCATCCCCAAATATGCCATGGTTCAAGGGATGGAAGGTGGA
GCGCAAAGAAGGCAACGCCTCCGGAGTGTCTCTGCTCGAGGCCCTGGACA
CCATTCTGCCCCCAACACGACCCACTGATAAGCCTCTGAGACTGCCACTG
CAAGACGTTTACAAAATTGGGGGAATTGGAACCGTGCCTGTGGGTCGGGT
GGAAACCGGAATCCTCAGACCCGGCATGGTGGTCACCTTCGCACCAGTGA
ATATAACGACAGAGGTCAAATCTGTGGAGATGCACCATGAGGCATTGAGC
GAGGCACTCCCAGGAGACAACGTGGGTTTCAACGTGAAAAATGTCTCAGT
TAAGGACATCCGACGCGGCAACGTGTGCGGAGATAGCAAATCTGACCCCC
CCCAGGAGGCCGCTCAATTCACAAGTCAGGTTATCATCCTTAATCACCCT
GGCCAAATATCTGCAGGCTACAGCCCCGTGATCGATTGTCACACAGCTCA
TATCGCCTGTAAATTTGCTGAACTCAAAGAAAAGATTGACCGCAGATCAG
GAAAAAAGCTGGAGGACAACCCTAAAAGTCTGAAGTCCGGCGACGCTGCC
ATCGTGGAGATGGTCCCTGGGAAACCCATGTGCGTGGAGTCCTTTTCTCA
GTACCCCCCTCTGGGACGATTCGCCGTGCGCGACATGAGACAGACTGTCG
CCGTGGGCGTCATTAAAAATGTGGAAAAAAAATCAGGAGGTGCAGGGAAA
GTGACAAAGAGTGCCCAGAAAGCACAGAAGGCTGGCAAGTGA(SEQ ID
NO: 8).

Optionally, the polynucleotide sequence encoding the vector genome may comprise a Kozak sequence, including but not limited to GCCACCATGG (SEQ ID NO: 10). Kozak sequence may overlap the polynucleotide sequence encoding an eEF1A2 protein or a functional variant thereof. For example, the vector genome may comprise a polynucleotide sequence (with Kozak underlined) at least 75%, 80%, 85%, 90%, 92%, 93%, 94%, 95%, 96%, 97%, 98%, 99%, or 100% identical to:

gccaccATGGGCAAGGAGAAGACCCACATCAACATCGTGGTCATCGGCCA
CGTGGACTCCGGAAAGTCCACCACCACGGGCCACCTCATCTACAAATGCG
GAGGTATTGACAAAAGGACCATTGAGAAGTTCGAGAAGGAGGCGGCTGAG
ATGGGGAAGGGATCCTTCAAGTATGCCTGGGTGCTGGACAAGCTGAAGGC
GGAGCGTGAGCGCGGCATCACCATCGACATCTCCCTCTGGAAGTTCGAGA
CCACCAAGTACTACATCACCATCATCGATGCCCCCGGCCACCGCGACTTC
ATCAAGAACATGATCACGGGTACATCCCAGGCGGACTGCGCAGTGCTGAT
CGTGGCGGCGGGCGTGGGCGAGTTCGAGGCGGGCATCTCCAAGAATGGGC
AGACGCGGGAGCATGCCCTGCTGGCCTACACGCTGGGTGTGAAGCAGCTC
ATCGTGGGCGTGAACAAAATGGACTCCACAGAGCCGGCCTACAGCGAGAA
GCGCTACGACGAGATCGTCAAGGAAGTCAGCGCCTACATCAAGAAGATCG
GCTACAACCCGGCCACCGTGCCCTTTGTGCCCATCTCCGGCTGGCACGGT
GACAACATGCTGGAGCCCTCCCCCAACATGCCGTGGTTCAAGGGCTGGAA
GGTGGAGCGTAAGGAGGGCAACGCAAGCGGCGTGTCCCTGCTGGAGGCCC
TGGACACCATCCTGCCCCCCACGCGCCCCACGGACAAGCCCCTGCGCCTG
CCGCTGCAGGACGTGTACAAGATTGGCGGCATTGGCACGGTGCCCGTGGG
CCGGGTGGAGACCGGCATCCTGCGGCCGGGCATGGTGGTGACCTTTGCGC
CAGTGAACATCACCACTGAGGTGAAGTCAGTGGAGATGCACCACGAGGCT
CTGAGCGAAGCTCTGCCCGGCGACAACGTCGGCTTCAATGTGAAGAACGT
GTCGGTGAAGGACATCCGGCGGGGCAACGTGTGTGGGGACAGCAAGTCTG
ACCCGCCGCAGGAGGCTGCTCAGTTCACCTCCCAGGTCATCATCCTGAAC
CACCCGGGGCAGATTAGCGCCGGCTACTCCCCGGTCATCGACTGCCACAC
AGCCCACATCGCCTGCAAGTTTGCGGAGCTGAAGGAGAAGATTGACCGGC
GCTCTGGCAAGAAGCTGGAGGACAACCCCAAGTCCCTGAAGTCTGGAGAC
GCGGCCATCGTGGAGATGGTGCCGGGAAAGCCCATGTGTGTGGAGAGCTT
CTCCCAGTACCCGCCTCTCGGCCGCTTCGCCGTGCGCGACATGAGGCAGA
CGGTGGCCGTAGGCGTCATCAAGAACGTGGAGAAGAAGAGCGGCGGCGCC
GGCAAGGTCACCAAGTCGGCGCAGAAGGCGCAGAAGGCGGGCAAG(SEQ
ID NO: 9).

In some embodiment, the Kozak sequence is an alternative Kozak sequence comprising or consisting of any one of:

(gcc)gccRccAUGG (SEQ ID NO: 11);

AGNNAUGN;

ANNAUGG;

ACCAUGG;

GACACCAUGG (SEQ ID NO: 12).

In some embodiments, the vector genome comprises no Kozak sequence.

### Vector Genome

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be from the same serotype as the capsid or a different serotype (e.g., AAV2 ITRs may be used).

In some embodiments, the 5′ ITR comprises a polynucleotide sequence at least 75%, 80%, 85%, 90%, 92%, 93%, 94%, 95%, 96%, 97%, 98%, 99%, or 100% identical to:

CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT(SEQ ID N
O: 18)

In some embodiments, the 5′ ITR comprises a polynucleotide sequence at least 75%, 80%, 85%, 90%, 92%, 93%, 94%, 95%, 96%, 97%, 98%, 99%, or 100% identical to:

GCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGA
GTGGCCAACTCCATCACTAGGGGTTCCTTGTAGTTAATGATTAACCCGCC
ATGCTACTTATCTACGTA(SEQ ID NO: 19)

In some embodiments, the 5′ ITR comprises a polynucleotide sequence at least 75%, 80%, 85%, 90%, 92%, 93%, 94%, 95%, 96%, 97%, 98%, 99%, or 100% identical to:

CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTA(SEQ ID NO: 20)

In some embodiments, the 3′ ITR comprises a polynucleotide sequence at least 75%, 80%, 85%, 90%, 92%, 93%, 94%, 95%, 96%, 97%, 98%, 99%, or 100% identical to:

AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG(SEQ ID N
O: 21)

In some embodiments, the 3′ ITR comprises a polynucleotide sequence at least 75%, 80%, 85%, 90%, 92%, 93%, 94%, 95%, 96%, 97%, 98%, 99%, or 100% identical to:

TACGTAGATAAGTAGCATGGCGGGTTAATCATTAACTACAAGGAACCCCT
AGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCA
GTGAGCGAGCGAGCGCGC(SEQ ID NO: 63)

In some embodiments the vector genome comprises one or more filler sequences, e.g., at least 75%, 80%, 85%, 90%, 92%, 93%, 94%, 95%, 96%, 97%, 98%, 99%, or 100% identical to:

GCGGCAATTCAGTCGATAACTATAACGGTCCTAAGGTAGCGATTTAAATA
CGCGCTCTCTTAAGGTAGCCCCGGGACGCGTCAATTGACTACAAACCGAG
TATCTGCAGAGGGCCCTGCGTATG (SEQ ID NO: 22);

CTTCTGAGGCGGAAAGAACCAGATCCTCTCTTAAGGTAGCATCGAGATTT
AAATTAGGGATAACAGGGTAATGGCGCGGGCCGC (SEQ ID NO: 23)
;

or

GTTACCCAGGCTGGAGTGCAGTGGCACATTTCTGCTCACTGCAACCTCCT
CCTCCCTGGGTTC (SEQ ID NO:24).

### Promoters

In some embodiments, the polynucleotide sequence encoding an eEF1A2 protein or functional variant thereof is operably linked to a promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least 75%, 80%, 85%, 90%, 92%, 93%, 94%, 95%, 96%, 97%, 98%, 99%, or 100% identical to:

ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCATGGTCGA
GGTGAGCCCCACGTTCTGCTTCACTCTCCCCATCTCCCCCCCCTCCCCAC
CCCCAATTTTGTATTTATTTATTTTTTAATTATTTTGTGCAGCGATGGGG
GCGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCG
CTCCGAAAGTTTCCTTTTATGGCGAGGCGGCGGCGGCGGCGGCCCTATAA
AAAGCGAAGCGCGCGGCGGGCGG(SEQ ID NO: 14)

In some embodiments, a polynucleotide sequence encoding an eEF1A2 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in a neuron to a greater extent than in a non-neuronal cell. In some embodiments, tissue-specific promoter is a selected from any various neuron-specific promoters including but not limited to hSYN1 (human synapsin), INA (alpha-internexin), NES (nestin), TH (tyrosine hydroxylase), FOXA2 (Forkhead box A2), CaMKII (calmodulin-dependent protein kinase II), and NSE (neuron-specific enolase). In some cases, the promoter is a ubiquitous promoter. A “ubiquitous promoter” refers to a promoter that is not tissue-specific under experimental or clinical conditions. In some cases, the ubiquitous promoter is any one of CMV, CAG, UBC, PGK, EF1-alpha, GAPDH, SV40, HBV, chicken beta-actin, and human beta-actin promoters.

In some embodiments, the promoter sequence is selected from Table 3. In some embodiments, the promoter comprises a polynucleotide sequence at least 75%, 80%, 85%, 90%, 92%, 93%, 94%, 95%, 96%, 97%, 98%, 99%, or 100% identical to any one of SEQ ID NOS 3, 14, 16-17, and 25-30.

TABLE 3

| PROMOTER | SEQ ID NO: |
|---|---|
| Human beta-actin (HuBa) | 25 |
| Chicken beta-actin (CBA) | 26 |
| Cytomegalovirus (CMV) | 16 |
| Cytomegalovirus (CMV) (second version) | 17 |
| CAG promoter | 14 |
| Human EF1-alpha (EF1-α) | 27 |
| Human Synapsin1 (Syn), short version | 28 |
| Human Synapsin1 (Syn) with 3′ extension | 3 |
| Human Synapsin1 (Syn) with 5′ extension | 29 |
| Human CamKIIa (CaMKIIa) | 30 |
| eSYN promoter | 64 |

In a preferred embodiment, the vector genome comprises a polynucleotide sequence at least 75%, 80%, 85%, 90%, 92%, 93%, 94%, 95%, 96%, 97%, 98%, 99%, or 100% identical to SEQ ID NO: 3.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

### Other Regulatory Elements

In some cases, vectors of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the vector comprises a CMV enhancer.

In certain embodiments, the vectors comprise one or more enhancers. In particular embodiments, the enhancer is a CMV enhancer sequence, a GAPDH enhancer sequence, a β-actin enhancer sequence, or an EF1-α enhancer sequence. Sequences of the foregoing are known in the art. For example, the sequence of the CMV immediate early (IE) enhancer is:

ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCA(SEQ ID
NO: 31)

In certain embodiments, the vectors comprise one or more introns. In particular embodiments, the intron is a rabbit globin intron sequence, a chicken β-actin intron sequence, a synthetic intron sequence, or an EF1-α intron sequence.

In certain embodiments, the vectors comprise a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the vectors comprise one or more transcript stabilizing element. In particular embodiments, the transcript stabilizing element is a WPRE sequence, a HPRE sequence, a scaffold-attachment region, a 3′ UTR, or a 5′ UTR. In particular embodiments, the vectors comprise both a 5′ UTR and a 3′ UTR.

In some embodiments, the vector comprises a 5′ untranslated region (UTR) selected from Table 4. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 32-40.

TABLE 4

| 5′ UNTRANSLATED REGION | SEQ ID NO: |
|---|---|
| Human beta-actin exon/intron | 32 |
| Chicken beta-actin exon/intron + rabbit globin intron | 33 |
| 5′ UTR-Syn1 Hs | 34 |
| CMV IE exon | 35 |
| TPL-eMLP (adenovirus derived enhancer element) | 36 |
| Human EF1-α intron/exon | 37 |
| Human EF1-α, intron A | 38 |
| 5′ UTR human CamKIIa | 39 |
| B-globin intron | 40 |

In some embodiments, the vector comprises a 3′ untranslated region selected from Table 5. In some embodiments, the vector genome comprises a polynucleotide sequence at least 75%, 80%, 85%, 90%, 92%, 93%, 94%, 95%, 96%, 97%, 98%, 99%, or 100% identical to any one of SEQ ID NOS 41-49.

TABLE 5

| 3′ UNTRANSLATED REGION | SEQ ID NO: |
|---|---|
| WPRE(x) (mutated woodchuck hepatitis regulatory element -version 1) | 41 |
| WPRE(x) (mutated woodchuck hepatitis regulatory element -version 2) | 42 |
| WPRE(x) (mutated woodchuck hepatitis regulatory element -version 3) | 43 |
| CAAX | 44 |
| EES | 45 |
| HPRE | 46 |
| R2V17 (HepB derived enhancer element) | 47 |
| 3′UTR(globin) | 48 |
| WPRE(r) | 49 |

In some embodiments, the vector comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 50-54.

TABLE 6

| POLYADENYLATION SITE | SEQ ID NO: |
|---|---|
| Rabbit globin (pAGlobin-Oc) | 50 |
| Bovine growth hormone (pAGH-Bt -version 1) | 51 |
| Bovine growth hormone (pAGH-Bt -version 2) | 52 |
| Bovine growth hormone (pAGH-Bt -version 3) | 53 |
| Human growth hormone (pAGH-Hs) | 54 |

Illustrative vector genomes are depicted in FIGS. 2-5 and provided as SEQ ID NOs: 55-58 or 65-68. In some embodiments, the vector genome comprises, consists essentially of, or consists of a polynucleotide sequence that shares at least 90%, 91%, 92%, 93%, 94%, 95%, 96%, 97%, 98%, 99%, or 100% identity to any one of SEQ ID NOs: 55-58 or 65-68, optionally with or without the ITR sequences in lowercase. The coding sequence is underlined. Alternative vector genome sequences are provided as SEQ ID NOs: 65-68.

### V1 - Vector Genome - 3,144 Bp (FIG. 2)

(SEQ ID NO: 55)cctgcaggcagctgcgcgctcgctcgctcactgag
gccgcccgggcaaagcccgggcgtcgggcgacctttggtcgcccggcctc
agtgagcgagcgagcgcgcagagagggagtggccaactccatcactaggg
gttcctGCGGCAATTCAGTCGATAACTATAACGGTCCTAAGGTAGCGATT
TAAATACGCGCTCTCTTAAGGTAGCCCCGGGACGCGTCAATTGACTACAA
ACCGAGTATCTGCAGAGGGCCCTGCGTATGAGTGCAAGTGGGTTTTAGGA
CCAGGATGAGGCGGGGTGGGGGTGCCTACCTGACGACCGACCCCGACCCA
CTGGACAAGCACCCAACCCCCATTCCCCAAATTGCGCATCCCCTATCAGA
GAGGGGGAGGGGAAACAGGATGCGGCGAGGCGCGTGCGCACTGCCAGCTT
CAGCACCGCGGACAGTGCCTTCGCCCCCGCCTGGCGGCGCGCGCCACCGC
CGCCTCAGCACTGAAGGCGCGCTGACGTCACTCGCCGGTCCCCCGCAAAC
TCCCCTTCCCGGCCACCTTGGTCGCGTCCGCGCCGCCGCCGGCCCAGCCG
GACCGCACCACGCGAGGCGCGAGATAGGGGGGCACGGGCGCGACCATCTG
CGCTGCGGCGCCGGCGACTCAGCGCTGCCTCAGTCTGCGGTGGGCAGCGG
AGGAGTCGTGTCGTGCCTGAGAGCGCAGATGGGCAAGGAGAAGACCCACA TCAACATCGTGGTCATCGGCCACGTGGACTCCGGAAAGTCCACCACCACG GGCCACCTCATCTACAAATGCGGAGGTATTGACAAAAGGACCATTGAGAA GTTCGAGAAGGAGGCGGCTGAGATGGGGAAGGGATCCTTCAAGTATGCCT GGGTGCTGGACAAGCTGAAGGCGGAGCGTGAGCGCGGCATCACCATCGAC ATCTCCCTCTGGAAGTTCGAGACCACCAAGTACTACATCACCATCATCGA TGCCCCCGGCCACCGCGACTTCATCAAGAACATGATCACGGGTACATCCC AGGCGGACTGCGCAGTGCTGATCGTGGCGGCGGGCGTGGGCGAGTTCGAG GCGGGCATCTCCAAGAATGGGCAGACGCGGGAGCATGCCCTGCTGGCCTA CACGCTGGGTGTGAAGCAGCTCATCGTGGGCGTGAACAAAATGGACTCCA CAGAGCCGGCCTACAGCGAGAAGCGCTACGACGAGATCGTCAAGGAAGTC AGCGCCTACATCAAGAAGATCGGCTACAACCCGGCCACCGTGCCCTTTGT GCCCATCTCCGGCTGGCACGGTGACAACATGCTGGAGCCCTCCCCCAACA TGCCGTGGTTCAAGGGCTGGAAGGTGGAGCGTAAGGAGGGCAACGCAAGC GGCGTGTCCCTGCTGGAGGCCCTGGACACCATCCTGCCCCCCACGCGCCC CACGGACAAGCCCCTGCGCCTGCCGCTGCAGGACGTGTACAAGATTGGCG GCATTGGCACGGTGCCCGTGGGCCGGGTGGAGACCGGCATCCTGCGGCCG GGCATGGTGGTGACCTTTGCGCCAGTGAACATCACCACTGAGGTGAAGTC AGTGGAGATGCACCACGAGGCTCTGAGCGAAGCTCTGCCCGGCGACAACG TCGGCTTCAATGTGAAGAACGTGTCGGTGAAGGACATCCGGCGGGGCAAC GTGTGTGGGGACAGCAAGTCTGACCCGCCGCAGGAGGCTGCTCAGTTCAC CTCCCAGGTCATCATCCTGAACCACCCGGGGCAGATTAGCGCCGGCTACT CCCCGGTCATCGACTGCCACACAGCCCACATCGCCTGCAAGTTTGCGGAG CTGAAGGAGAAGATTGACCGGCGCTCTGGCAAGAAGCTGGAGGACAACCC CAAGTCCCTGAAGTCTGGAGACGCGGCCATCGTGGAGATGGTGCCGGGAA AGCCCATGTGTGTGGAGAGCTTCTCCCAGTACCCGCCTCTCGGCCGCTTC GCCGTGCGCGACATGAGGCAGACGGTGGCCGTAGGCGTCATCAAGAACGT GGAGAAGAAGAGCGGCGGCGCCGGCAAGGTCACCAAGTCGGCGCAGAAGG CGCAGAAGGCGGGCAAGTGAAATCAACCTCTGGATTACAAAATTTGTGAA
AGATTGACTGGTATTCTTAACTATGTTGCTCCTTTTACGCTATGTGGATA
CGCTGCTTTAATGCCTTTGTATCATGCTATTGCTTCCCGTATGGCTTTCA
TTTTCTCCTCCTTGTATAAATCCTGGTTGCTGTCTCTTTATGAGGAGTTG
TGGCCCGTTGTCAGGCAACGTGGCGTGGTGTGCACTGTGTTTGCTGACGC
AACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGC
CTTGCCCGCTGCTGGACAGGGGCTCGGCTGTTGGGCACTGACAATTCCGT
GGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCT
TCCGCGTCTTCGCCTTCGCCCTCAGACGAGTCGGATCTCCCTTTGGGCCG
CCTCCCCGCCTGTGCCTTCTAGTTGCCAGCCATCTGTTGTTTGCCCCTCC
CCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTC
TGGGGGGTGGGGTGGGGCAGGACAGCAAGGGGGAGGATTGGGAAGACAAT
AGCAGGCATGCTGGGGATGCGGTGGGCTCTATGGCTTCTGAGGCGGAAAG
AACCAGATCCTCTCTTAAGGTAGCATCGAGATTTAAATTAGGGATAACAG
GGTAATGGCGCGGGCCGCaggaacccctagtgatggagttggccactccc
tctctgcgcgctcgctcgctcactgaggccgggcgaccaaaggtcgcccg
acgcccgggctttgcccgggcggcctcagtgagcgagcgagcgcgcagct
gcctgcagg

### V2 - Vector Genome - 3,035 Bp (FIG. 3)

(SEQ ID NO: 56)gcgcgctcgctcgctcactgaggccgcccgggcaa
agcccgggcgtcgggcgacctttggtcgcccggcctcagtgagcgagcga
gcgcgcagagagggagtggccaactccatcactaggggttccttgtagtt
aatgattaacccgccatgctacttatctacgtaAGTGCAAGTGGGTTTTA
GGACCAGGATGAGGCGGGGTGGGGGTGCCTACCTGACGACCGACCCCGAC
CCACTGGACAAGCACCCAACCCCCATTCCCCAAATTGCGCATCCCCTATC
AGAGAGGGGGAGGGGAAACAGGATGCGGCGAGGCGCGTGCGCACTGCCAG
CTTCAGCACCGCGGACAGTGCCTTCGCCCCCGCCTGGCGGCGCGCGCCAC
CGCCGCCTCAGCACTGAAGGCGCGCTGACGTCACTCGCCGGTCCCCCGCA
AACTCCCCTTCCCGGCCACCTTGGTCGCGTCCGCGCCGCCGCCGGCCCAG
CCGGACCGCACCACGCGAGGCGCGAGATAGGGGGGCACGGGCGCGACCAT
CTGCGCTGCGGCGCCGGCGACTCAGCGCTGCCTCAGTCTGCGGTGGGCAG
CGGAGGAGTCGTGTCGTGCCTGAGAGCGCAGGCCACCATGGGCAAGGAGA AGACCCACATCAACATCGTGGTCATCGGCCACGTGGACTCCGGAAAGTCC ACCACCACGGGCCACCTCATCTACAAATGCGGAGGTATTGACAAAAGGAC CATTGAGAAGTTCGAGAAGGAGGCGGCTGAGATGGGGAAGGGATCCTTCA AGTATGCCTGGGTGCTGGACAAGCTGAAGGCGGAGCGTGAGCGCGGCATC ACCATCGACATCTCCCTCTGGAAGTTCGAGACCACCAAGTACTACATCAC CATCATCGATGCCCCCGGCCACCGCGACTTCATCAAGAACATGATCACGG GTACATCCCAGGCGGACTGCGCAGTGCTGATCGTGGCGGCGGGCGTGGGC GAGTTCGAGGCGGGCATCTCCAAGAATGGGCAGACGCGGGAGCATGCCCT GCTGGCCTACACGCTGGGTGTGAAGCAGCTCATCGTGGGCGTGAACAAAA TGGACTCCACAGAGCCGGCCTACAGCGAGAAGCGCTACGACGAGATCGTC AAGGAAGTCAGCGCCTACATCAAGAAGATCGGCTACAACCCGGCCACCGT GCCCTTTGTGCCCATCTCCGGCTGGCACGGTGACAACATGCTGGAGCCCT CCCCCAACATGCCGTGGTTCAAGGGCTGGAAGGTGGAGCGTAAGGAGGGC AACGCAAGCGGCGTGTCCCTGCTGGAGGCCCTGGACACCATCCTGCCCCC CACGCGCCCCACGGACAAGCCCCTGCGCCTGCCGCTGCAGGACGTGTACA AGATTGGCGGCATTGGCACGGTGCCCGTGGGCCGGGTGGAGACCGGCATC CTGCGGCCGGGCATGGTGGTGACCTTTGCGCCAGTGAACATCACCACTGA GGTGAAGTCAGTGGAGATGCACCACGAGGCTCTGAGCGAAGCTCTGCCCG GCGACAACGTCGGCTTCAATGTGAAGAACGTGTCGGTGAAGGACATCCGG CGGGGCAACGTGTGTGGGGACAGCAAGTCTGACCCGCCGCAGGAGGCTGC TCAGTTCACCTCCCAGGTCATCATCCTGAACCACCCGGGGCAGATTAGCG CCGGCTACTCCCCGGTCATCGACTGCCACACAGCCCACATCGCCTGCAAG TTTGCGGAGCTGAAGGAGAAGATTGACCGGCGCTCTGGCAAGAAGCTGGA GGACAACCCCAAGTCCCTGAAGTCTGGAGACGCGGCCATCGTGGAGATGG TGCCGGGAAAGCCCATGTGTGTGGAGAGCTTCTCCCAGTACCCGCCTCTC GGCCGCTTCGCCGTGCGCGACATGAGGCAGACGGTGGCCGTAGGCGTCAT CAAGAACGTGGAGAAGAAGAGCGGCGGCGCCGGCAAGGTCACCAAGTCGG CGCAGAAGGCGCAGAAGGCGGGCAAGTGATCAACCTCTGGATTACAAAAT
TTGTGAAAGATTGACTGGTATTCTTAACTATGTTGCTCCTTTTACGCTAT
GTGGATACGCTGCTTTAATGCCTTTGTATCATGCTATTGCTTCCCGTATG
GCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTT
TCCGGGACTTTCGCTTTCCCCCTCCCTATTGCCACGGCGGAACTCATCGC
CGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTT
TGGGCCGCCTCCCCGCACTGCCCGGGTGGCATCCCTGTGACCCCTCCCCA
GTGCCTCTCCTGGCCCTGGAAGTTGCCACTCCAGTGCCCACCAGCCTTGT
CCTAATAAAATTAAGTTGCATCATTTTGTCTGACTAGGTGTCCTTCTATA
ATATTATGGGGTGGAGGGGGGTGGTATGGAGCAAGGGGCCCAAGTTGGGA
AGAAACCTGTAGGGCCTGCGTTACCCAGGCTGGAGTGCAGTGGCACATTT
CTGCTCACTGCAACCTCCTCCTCCCTGGGTTCtacgtagataagtagcat
ggcgggttaatcattaactacaaggaacccctagtgatggagttggccac
tccctctctgcgcgctcgctcgctcactgaggccgggcgaccaaaggtcg
cccgacgcccgggctttgcccgggcggcctcagtgagcgagcgagcgcgc

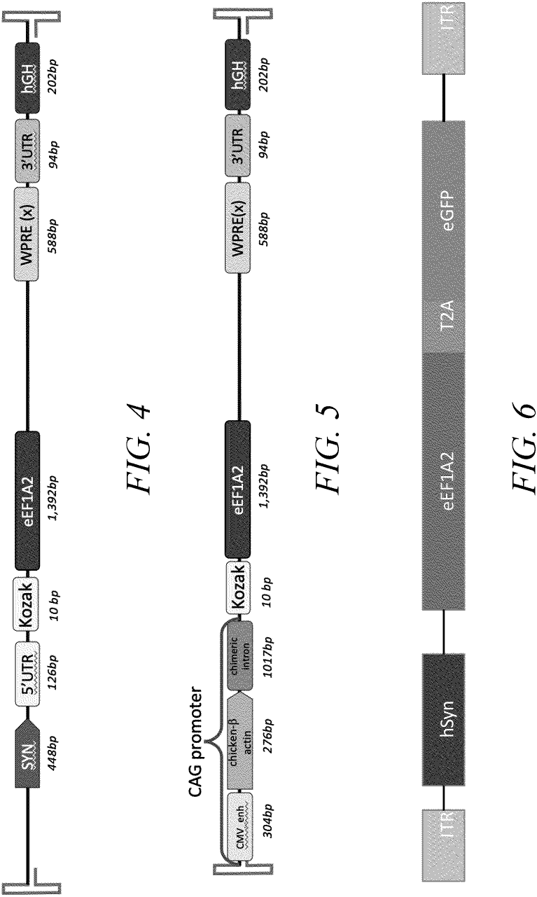

### V3 - Vector Genome - 3,263 Bp (FIG. 4)

(SEQ ID NO: 57)gcgcgctcgctcgctcactgaggccgcccgggcaa
agcccgggcgtcgggcgacctttggtcgcccggcctcagtgagcgagcga
gcgcgcagagagggagtggccaactccatcactaggggttccttgtagtt
aatgattaacccgccatgctacttatctacgtaAGTGCAAGTGGGTTTTA
GGACCAGGATGAGGCGGGGTGGGGGTGCCTACCTGACGACCGACCCCGAC
CCACTGGACAAGCACCCAACCCCCATTCCCCAAATTGCGCATCCCCTATC
AGAGAGGGGGAGGGGAAACAGGATGCGGCGAGGCGCGTGCGCACTGCCAG
CTTCAGCACCGCGGACAGTGCCTTCGCCCCCGCCTGGCGGCGCGCGCCAC
CGCCGCCTCAGCACTGAAGGCGCGCTGACGTCACTCGCCGGTCCCCCGCA
AACTCCCCTTCCCGGCCACCTTGGTCGCGTCCGCGCCGCCGCCGGCCCAG
CCGGACCGCACCACGCGAGGCGCGAGATAGGGGGGCACGGGCGCGACCAT
CTGCGCTGCGGCGCCGGCGACTCAGCGCTGCCTCAGTCTGCGGTGGGCAG
CGGAGGAGTCGTGTCGTGCCTGAGAGCGCAGAGTCTGCGGTGGGCAGCGG
AGGAGTCGTGTCGTGCCTGAGAGCGCAGCTGTGCTCCTGGGCACCGCGCA
GTCCGCCCCCGCGGCTCCTGGCCAGACCACCCCTAGGACCCCCTGCCCCA
AGTCGCAGCCACCATGGGCAAGGAGAAGACCCACATCAACATCGTGGTCA TCGGCCACGTGGACTCCGGAAAGTCCACCACCACGGGCCACCTCATCTAC AAATGCGGAGGTATTGACAAAAGGACCATTGAGAAGTTCGAGAAGGAGGC GGCTGAGATGGGGAAGGGATCCTTCAAGTATGCCTGGGTGCTGGACAAGC TGAAGGCGGAGCGTGAGCGCGGCATCACCATCGACATCTCCCTCTGGAAG TTCGAGACCACCAAGTACTACATCACCATCATCGATGCCCCCGGCCACCG CGACTTCATCAAGAACATGATCACGGGTACATCCCAGGCGGACTGCGCAG TGCTGATCGTGGCGGCGGGCGTGGGCGAGTTCGAGGCGGGCATCTCCAAG AATGGGCAGACGCGGGAGCATGCCCTGCTGGCCTACACGCTGGGTGTGAA GCAGCTCATCGTGGGCGTGAACAAAATGGACTCCACAGAGCCGGCCTACA GCGAGAAGCGCTACGACGAGATCGTCAAGGAAGTCAGCGCCTACATCAAG AAGATCGGCTACAACCCGGCCACCGTGCCCTTTGTGCCCATCTCCGGCTG GCACGGTGACAACATGCTGGAGCCCTCCCCCAACATGCCGTGGTTCAAGG GCTGGAAGGTGGAGCGTAAGGAGGGCAACGCAAGCGGCGTGTCCCTGCTG GAGGCCCTGGACACCATCCTGCCCCCCACGCGCCCCACGGACAAGCCCCT GCGCCTGCCGCTGCAGGACGTGTACAAGATTGGCGGCATTGGCACGGTGC CCGTGGGCCGGGTGGAGACCGGCATCCTGCGGCCGGGCATGGTGGTGACC TTTGCGCCAGTGAACATCACCACTGAGGTGAAGTCAGTGGAGATGCACCA CGAGGCTCTGAGCGAAGCTCTGCCCGGCGACAACGTCGGCTTCAATGTGA AGAACGTGTCGGTGAAGGACATCCGGCGGGGCAACGTGTGTGGGGACAGC AAGTCTGACCCGCCGCAGGAGGCTGCTCAGTTCACCTCCCAGGTCATCAT CCTGAACCACCCGGGGCAGATTAGCGCCGGCTACTCCCCGGTCATCGACT GCCACACAGCCCACATCGCCTGCAAGTTTGCGGAGCTGAAGGAGAAGATT GACCGGCGCTCTGGCAAGAAGCTGGAGGACAACCCCAAGTCCCTGAAGTC TGGAGACGCGGCCATCGTGGAGATGGTGCCGGGAAAGCCCATGTGTGTGG AGAGCTTCTCCCAGTACCCGCCTCTCGGCCGCTTCGCCGTGCGCGACATG AGGCAGACGGTGGCCGTAGGCGTCATCAAGAACGTGGAGAAGAAGAGCGG CGGCGCCGGCAAGGTCACCAAGTCGGCGCAGAAGGCGCAGAAGGCGGGCA AGTGATCAACCTCTGGATTACAAAATTTGTGAAAGATTGACTGGTATTCT
TAACTATGTTGCTCCTTTTACGCTATGTGGATACGCTGCTTTAATGCCTT
TGTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGCA
ACGTGGCGTGGTGTGCACTGTGTTTGCTGACGCAACCCCCACTGGTTGGG
GCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAAT
CATCGTCCTTTCCTTGGCTGCTCGCCTGTGTTGCCACCTGGATTCTGCGC
GGGACGTCCTTCTGCTACGTCCCTTCGGCCCTCAATCCAGCGGACCTTCC
TTCCCGCGGCCTGCTGCCGGCTCTGCGGCCTCTTCCGCGTCTTCGCCTTC
GCCCTCAGACGAGTCGGATCTCCCTTTGGGCCGCCTCCCCGCAGCTGGAG
CCTCGGTAGCCGTTCCTCCTGCCCGCTGGGCCTCCCAACGGGCCCTCCTC
CCCTCCTTGCACCGGCCCTTCCTGGTCTTTGAATAAATTCATTGCCTGCC
CGGGTGGCATCCCTGTGACCCCTCCCCAGTGCCTCTCCTGGCCCTGGAAG
TTGCCACTCCAGTGCCCACCAGCCTTGTCCTAATAAAATTAAGTTGCATC
ATTTTGTCTGACTAGGTGTCCTTCTATAATATTATGGGGTGGAGGGGGGT
GGTATGGAGCAAGGGGCCCAAGTTGGGAAGAAACCTGTAGGGCCTGCGTT
ACCCAGGCTGGAGTGCAGTGGCACATTTCTGCTCACTGCAACCTCCTCCT
CCCTGGGTTCtacgtagataagtagcatggcgggttaatcattaactaca
aggaacccctagtgatggagttggccactccctctctgcgcgctcgctcg
ctcactgaggccgggcgaccaaaggtcgcccgacgcccgggctttgcccg
ggcggcctcagtgagcgagcgagcgcgc

### V4 - Vector Genome - 4,299 Bp (FIG. 5)

(SEQ ID NO: 58)gcgcgctcgctcgctcactgaggccgcccgggcaa
agcccgggcgtcgggcgacctttggtcgcccggcctcagtgagcgagcga
gcgcgcagagagggagtggccaactccatcactaggggttccttgtagtt
aatgattaacccgccatgctacttatctacgtaCTCTGGAGACGCGTTAC
ATAACTTACGGTAAATGGCCCGCCTGGCTGACCGCCCAACGACCCCCGCC
CATTGACGTCAATAATGACGTATGTTCCCATAGTAACGCCAATAGGGACT
TTCCATTGACGTCAATGGGTGGAGTATTTACGGTAAACTGCCCACTTGGC
AGTACATCAAGTGTATCATATGCCAAGTACGCCCCCTATTGACGTCAATG
ACGGTAAATGGCCCGCCTGGCATTATGCCCAGTACATGACCTTATGGGAC
TTTCCTACTTGGCAGTACATCTACGTATTAGTCATCGCTATTACCATGGT
CGAGGTGAGCCCCACGTTCTGCTTCACTCTCCCCATCTCCCCCCCCTCCC
CACCCCCAATTTTGTATTTATTTATTTTTTAATTATTTTGTGCAGCGATG
GGGGCGGGGGGGGGGGGGGCGCGCGCCAGGCGGGGCGGGGCGGGGCGAGG
GGCGGGGCGGGGCGAGGCGGAGAGGTGCGGCGGCAGCCAATCAGAGCGGC
GCGCTCCGAAAGTTTCCTTTTATGGCGAGGCGGCGGCGGCGGCGGCCCTA
TAAAAAGCGAAGCGCGCGGCGGGCGGGAGTCGCTGCGCGCTGCCTTCGCC
CCGTGCCCCGCTCCGCCGCCGCCTCGCGCCGCCCGCCCCGGCTCTGACTG
ACCGCGTTACTCCCACAGGTGAGCGGGCGGGACGGCCCTTCTCCTCCGGG
CTGTAATTAGCGCTTGGTTTAATGACGGCTTGTTTCTTTTCTGTGGCTGC
GTGAAAGCCTTGAGGGGCTCCGGGAGGGCCCTTTGTGCGGGGGGAGCGGC
TCGGGGGGTGCGTGCGTGTGTGTGTGCGTGGGGAGCGCCGCGTGCGGCTC
CGCGCTGCCCGGCGGCTGTGAGCGCTGCGGGCGCGGCGCGGGGCTTTGTG
CGCTCCGCAGTGTGCGCGAGGGGAGCGCGGCCGGGGGCGGTGCCCCGCGG
TGCGGGGGGGGCTGCGAGGGGAACAAAGGCTGCGTGCGGGGTGTGTGCGT
GGGGGGGTGAGCAGGGGGTGTGGGCGCGTCGGTCGGGCTGCAACCCCCCC
TGCACCCCCCTCCCCGAGTTGCTGAGCACGGCCCGGCTTCGGGTGCGGGG
CTCCGTACGGGGCGTGGCGCGGGGCTCGCCGTGCCGGGCGGGGGGTGGCG
GCAGGTGGGGGTGCCGGGCGGGGCGGGGCCGCCTCGGGCCGGGGAGGGCT
CGGGGGAGGGGCGCGGCGGCCCCCGGAGCGCCGGCGGCTGTCGAGGCGCG
GCGAGCCGCAGCCATTGCCTTTTATGGTAATCGTGCGAGAGGGCGCAGGG
ACTTCCTTTGTCCCAAATCTGTGCGGAGCCGAAATCTGGGAGGCGCCGCC
GCACCCCCTCTAGCGGGCGCGGGGCGAAGCGGTGCGGCGCCGGCAGGAAG
GAAATGGGCGGGGAGGGCCTTCGTGCGTCGCCGCGCCGCCGTCCCCTTCT
CCCTCTCCAGCCTCGGGGCTGTCCGCGGGGGGACGGCTGCCTTCGGGGGG
GACGGGGCAGGGCGGGGTTCGGCTTCTGGCGTGTGACCGGCGGCTCTAGA
GCCTCTGCTAACCATGTTCATGCCTTCTTCTTTTTCCTACAGCGCCACCA TGGGCAAGGAGAAGACCCACATCAACATCGTGGTCATCGGCCACGTGGAC TCCGGAAAGTCCACCACCACGGGCCACCTCATCTACAAATGCGGAGGTAT TGACAAAAGGACCATTGAGAAGTTCGAGAAGGAGGCGGCTGAGATGGGGA AGGGATCCTTCAAGTATGCCTGGGTGCTGGACAAGCTGAAGGCGGAGCGT GAGCGCGGCATCACCATCGACATCTCCCTCTGGAAGTTCGAGACCACCAA GTACTACATCACCATCATCGATGCCCCCGGCCACCGCGACTTCATCAAGA ACATGATCACGGGTACATCCCAGGCGGACTGCGCAGTGCTGATCGTGGCG GCGGGCGTGGGCGAGTTCGAGGCGGGCATCTCCAAGAATGGGCAGACGCG GGAGCATGCCCTGCTGGCCTACACGCTGGGTGTGAAGCAGCTCATCGTGG GCGTGAACAAAATGGACTCCACAGAGCCGGCCTACAGCGAGAAGCGCTAC GACGAGATCGTCAAGGAAGTCAGCGCCTACATCAAGAAGATCGGCTACAA CCCGGCCACCGTGCCCTTTGTGCCCATCTCCGGCTGGCACGGTGACAACA TGCTGGAGCCCTCCCCCAACATGCCGTGGTTCAAGGGCTGGAAGGTGGAG CGTAAGGAGGGCAACGCAAGCGGCGTGTCCCTGCTGGAGGCCCTGGACAC CATCCTGCCCCCCACGCGCCCCACGGACAAGCCCCTGCGCCTGCCGCTGC AGGACGTGTACAAGATTGGCGGCATTGGCACGGTGCCCGTGGGCCGGGTG GAGACCGGCATCCTGCGGCCGGGCATGGTGGTGACCTTTGCGCCAGTGAA CATCACCACTGAGGTGAAGTCAGTGGAGATGCACCACGAGGCTCTGAGCG AAGCTCTGCCCGGCGACAACGTCGGCTTCAATGTGAAGAACGTGTCGGTG AAGGACATCCGGCGGGGCAACGTGTGTGGGGACAGCAAGTCTGACCCGCC GCAGGAGGCTGCTCAGTTCACCTCCCAGGTCATCATCCTGAACCACCCGG GGCAGATTAGCGCCGGCTACTCCCCGGTCATCGACTGCCACACAGCCCAC ATCGCCTGCAAGTTTGCGGAGCTGAAGGAGAAGATTGACCGGCGCTCTGG CAAGAAGCTGGAGGACAACCCCAAGTCCCTGAAGTCTGGAGACGCGGCCA TCGTGGAGATGGTGCCGGGAAAGCCCATGTGTGTGGAGAGCTTCTCCCAG TACCCGCCTCTCGGCCGCTTCGCCGTGCGCGACATGAGGCAGACGGTGGC CGTAGGCGTCATCAAGAACGTGGAGAAGAAGAGCGGCGGCGCCGGCAAGG TCACCAAGTCGGCGCAGAAGGCGCAGAAGGCGGGCAAGTGATCAACCTCT
GGATTACAAAATTTGTGAAAGATTGACTGGTATTCTTAACTATGTTGCTC
CTTTTACGCTATGTGGATACGCTGCTTTAATGCCTTTGTATCATGCTATT
GCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACC
TGTCAGCTCCTTTCCGGGACTTTCGCTTTCCCCCTCCCTATTGCCACGGC
GGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCT
TGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGC
TGCCGGCTCTGCGGCCTCTTCCGCGTCTTCGCCTTCGCCCTCAGACGAGT
CGGATCTCCCTTTGGGCCGCCTCCCCGCAGCTGGAGCCTCGGTAGCCGTT
CCTCCTGCCCGCTGGGCCTCCCAACGGGCCCTCCTCCCCTCCTTGCACCG
GCCCTTCCTGGTCTTTGAATAAATTCATTGCCTGCCCGGGTGGCATCCCT
GTGACCCCTCCCCAGTGCCTCTCCTGGCCCTGGAAGTTGCCACTCCAGTG
CCCACCAGCCTTGTCCTAATAAAATTAAGTTGCATCATTTTGTCTGACTA
GGTGTCCTTCTATAATATTATGGGGTGGAGGGGGGTGGTATGGAGCAAGG
GGCCCAAGTTGGGAAGAAACCTGTAGGGCCTGCGTTACCCAGGCTGGAGT
GCAGTGGCACATTTCTGCTCACTGCAACCTCCTCCTCCCTGGGTTCtacg
tagataagtagcatggcgggttaatcattaactacaaggaacccctagtg
atggagttggccactccctctctgcgcgctcgctcgctcactgaggccgg
gcgaccaaaggtcgcccgacgcccgggctttgcccgggcggcctcagtga
gcgagcgagcgcgc

In an embodiment, the expression cassette comprises, in 5′ to 3′ order, HuBA promoter, the polynucleotide sequence encoding eEF1A2 or a functional variant thereof, WPRE(x), and pAGlobin-Oc.

In an embodiment, the expression cassette comprises, in 5′ to 3′ order, CMV promoter, TPL-eMLP enhancer, the polynucleotide sequence encoding eEF1A2 or a functional variant thereof, WPRE(r), and pAGlobin-Oc.

In an embodiment, the expression cassette comprises, in 5′ to 3′ order, Syn promoter, the polynucleotide sequence encoding eEF1A2 or a functional variant thereof, WPRE(r), 3′UTR (globin), and pAGH-Bt.

In an embodiment, the expression cassette comprises, in 5′ to 3′ order, CBA promoter, the polynucleotide sequence encoding eEF1A2 or a functional variant thereof, and pAGH-Bt.

In an embodiment, the expression cassette comprises, in 5′ to 3′ order, EF1α promoter, the polynucleotide sequence encoding eEF1A2 or a functional variant thereof, and pAGlobin-Oc.

In an embodiment, the expression cassette comprises, in 5′ to 3′ order, HuBA promoter, the polynucleotide sequence encoding eEF1A2 or a functional variant thereof, R2V17, and pAGH-Bt.

In an embodiment, the expression cassette comprises, in 5′ to 3′ order, Syn promoter, the polynucleotide sequence encoding eEF1A2 or a functional variant thereof, WPRE(x), 3′UTR (globin), and pAGH-Hs.

In an embodiment, the expression cassette comprises, in 5′ to 3′ order, CaMKIIa promoter, the polynucleotide sequence encoding eEF1A2 or a functional variant thereof, WPRE(r), and pAGH-Hs.

In an embodiment, the expression cassette comprises, in 5′ to 3′ order, CMV promoter, TPL-eMLP enhancer, the polynucleotide sequence encoding eEF1A2 or a functional variant thereof, WPRE(r), and pAGH-Hs.

In an embodiment, the expression cassette comprises, in 5′ to 3′ order, HuBA promoter, the polynucleotide sequence encoding eEF1A2 or a functional variant thereof, and pAGH-Hs.

In an embodiment, the expression cassette comprises, in 5′ to 3′ order, CMV promoter, TPL/eMLP enhancer, the polynucleotide sequence encoding eEF1A2 or a functional variant thereof, R2V17, 3′UTR (globin), and pAGH-Bt.

In an embodiment, the expression cassette comprises, in 5′ to 3′ order, EF1α promoter, the polynucleotide sequence encoding eEF1A2 or a functional variant thereof, WPRE(r), and pAGH-Bt.

In an embodiment, the expression cassette comprises, in 5′ to 3′ order, Syn promoter, the polynucleotide sequence encoding eEF1A2 or a functional variant thereof, R2V17, and pAGlobin-Oc.

In an embodiment, the expression cassette comprises, in 5′ to 3′ order, CaMKIIa promoter, the polynucleotide sequence encoding eEF1A2 or a functional variant thereof, R2V17, and pAGlobin-Oc.

In an embodiment, the expression cassette comprises, in 5′ to 3′ order, CBA promoter, the polynucleotide sequence encoding eEF1A2 or a functional variant thereof, WPRE(x), 3′UTR (globin), and pAGH-Hs.

In an embodiment, the expression cassette comprises, in 5′ to 3′ order, CBA promoter, the polynucleotide sequence encoding eEF1A2 or a functional variant thereof, 3′UTR (globin), and pAGlobin-Oc.

In an embodiment, the expression cassette comprises, in 5′ to 3′ order, CaMKIIa promoter, the polynucleotide sequence encoding eEF1A2 or a functional variant thereof, R2V17, and pAGH-Bt.

In an embodiment, the expression cassette comprises, in 5′ to 3′ order, EF1α promoter, the polynucleotide sequence encoding eEF1A2 or a functional variant thereof, R2V17, 3′UTR (globin), and pAGH-Hs.

In an embodiment, the expression cassette comprises, in 5′ to 3′ order, CMV promoter, the polynucleotide sequence encoding eEF1A2 or a functional variant thereof, R2V17, 3′UTR (globin), and pAGH-Hs.

In an embodiment, the expression cassette comprises, in 5′ to 3′ order, CMV promoter, the polynucleotide sequence encoding eEF1A2 or a functional variant thereof, and pAGH-Hs.

In an embodiment, the expression cassette comprises, in 5′ to 3′ order, hSYN promoter, the polynucleotide sequence encoding eEF1A2 or a functional variant thereof, WPRE(x), and pAGH-Bt.

In an embodiment, the expression cassette comprises, in 5′ to 3′ order, hSYN promoter, the polynucleotide sequence encoding eEF1A2 or a functional variant thereof, WPRE(x), and pAGH-Hs.

In an embodiment, the expression cassette comprises, in 5′ to 3′ order, hSYN promoter, Kozak, the polynucleotide sequence encoding eEF1A2 or a functional variant thereof, WPRE(x), and pAGH-Hs.

In an embodiment, the expression cassette comprises, in 5′ to 3′ order, CAG promoter, the polynucleotide sequence encoding eEF1A2 or a functional variant thereof, WPRE(x), and pAGH-Hs.

In an embodiment, the expression cassette comprises, in 5′ to 3′ order, CAG promoter, Kozak, the polynucleotide sequence encoding eEF1A2 or a functional variant thereof, WPRE(x), and pAGH-Hs.

In an embodiment, the expression cassette comprises, in 5′ to 3′ order, hSYN promoter, the polynucleotide sequence encoding eEF1A2 or a functional variant thereof, WPRE(x), and pAGH-Bt.

In an embodiment, the expression cassette comprises, in 5′ to 3′ order, hSYN promoter, the polynucleotide sequence encoding eEF1A2 or a functional variant thereof, and pAGH-Hs.

In an embodiment, the expression cassette comprises, in 5′ to 3′ order, hSYN promoter, Kozak, the polynucleotide sequence encoding eEF1A2 or a functional variant thereof, and pAGH-Hs.

In an embodiment, the expression cassette comprises, in 5′ to 3′ order, CAG promoter, the polynucleotide sequence encoding eEF1A2 or a functional variant thereof, and pAGH-Hs.

In an embodiment, the expression cassette comprises, in 5′ to 3′ order, CAG promoter, Kozak, the polynucleotide sequence encoding eEF1A2 or a functional variant thereof, and pAGH-Hs.

### ADENO-ASSOCIATED VIRUS VECTOR

Adeno-associated virus (AAV) is a replication-deficient parvovirus, the single-stranded DNA genome of which is about 4.7 kb in length including two ~145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV- 10, AAV-11, AAV- 12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6kb), rAAV comprising a self-complementary genome can only hold about half of that amount (≈2.4 kb).

In other cases, the rAAV vector comprises a single stranded genome. As defined herein, a “single standard” genome refers to a genome that is not self-complementary. In most cases, non-recombinant AAVs are have singled stranded DNA genomes. There have been some indications that rAAVs should be scAAVs to achieve efficient transduction of cells. The present disclosure contemplates, however, rAAV vectors that maybe have singled stranded genomes, rather than self-complementary genomes, with the understanding that other genetic modifications of the rAAV vector may be beneficial to obtain optimal gene transcription in target cells. In some cases, the present disclosure relates to single-stranded rAAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In some cases, the rAAV vector is of the serotype AAV1, AAV2, AAV4, AAV5, AAV6, AAV7, AAV8, AAV9, AAV10, AAV11, AAV12, AAV13, AAVrh10, or AAVrh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some cases, the rAAV vector is of the serotype AAV9. In some embodiments, said rAAV vector is of serotype AAV9 and comprises a single stranded genome. In some embodiments, said rAAV vector is of serotype AAV9 and comprises a self-complementary genome. In some embodiments, a rAAV vector comprises the inverted terminal repeat (ITR) sequences of AAV2. In some embodiments, the rAAV vector comprises an AAV2 genome, such that the rAAV vector is an AAV-2/9 vector, an AAV-2/6 vector, or an AAV-2/8 vector.

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, e.g., substitutions, deletions, or insertions, within a capsid protein, e.g., VP1, VP2 and/or VP3. In particular embodiments, the modification provides for reduced immunogenicity when the AAV vector is provided to a subject.

Capsid proteins of a rAAV may be modified so that the rAAV is targeted to a particular target tissue of interest such as neurons or more particularly a dopaminergic neuron. See, for example, Albert et al. AAV Vector-Mediated Gene Delivery to Substantia Nigra Dopamine Neurons: Implications for Gene Therapy and Disease Models. Genes. 2017 Feb 8; see also U.S. Pat. No. 6,180,613 and U.S. Pat. Pub. No. US20120082650A1, the disclosures of both of which are incorporated by reference herein. In some embodiments, the rAAV is directly injected into the substantia nigra of the subject.

In some embodiments, the rAAV virion is an AAV2 rAAV virion. The capsid many be an AAV2 capsid or functional variant thereof. In some embodiments, the AAV2 capsid shares at least 98%, 99%, or 100% identity to a reference AAV2 capsid, e.g.,

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL(SEQ ID NO: 59)

In some embodiments, the rAAV virion is an AAV9 rAAV virion. The capsid many be an AAV9 capsid or functional variant thereof. In some embodiments, the AAV9 capsid shares at least 98%, 99%, or 100% identity to a reference AAV9 capsid, e.g.,

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL(SEQ ID NO: 15
)

In some embodiments, the rAAV virion is an AAV-PHP.B rAAV virion or a neutrotrophic variant thereof, such as, without limitation, those disclosed in Int′l Pat. Pub. Nos. WO 2015/038958 A1and WO 2017/100671 A1. For example, the AAV capsid may comprise at least 4 contiguous amino acids from the sequence TLAVPFK (SEQ ID NO:61) or KFPVALT (SEQ ID NO:62), e.g., inserted between a sequence encoding for amino acids 588 and 589 of AAV9.

The capsid many be an AAV-PHP.B capsid or functional variant thereof. In some embodiments, the AAV-PHP.B capsid shares at least 98%, 99%, or 100% identity to a reference AAV-PHP.B capsid, e.g.,

MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQTLAVPFKAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL(SEQ ID
NO: 60)

Further AAV capsids used in the rAAV virions of the disclosure include those disclosed in Pat. Pub. Nos. WO 2009/012176 A2and WO 2015/168666 A2.

### Pharmaceutical Compositions and Kits

In an aspect, the disclosure provides pharmaceutical compositions comprising the rAAV virion of the disclosure and one or more pharmaceutically acceptable carriers, diluents, or excipients.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Pluronic™ F-68 at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

### Methods of Use

In an aspect, the disclosure provides a method of increasing eEF1A2 activity in a cell, comprising contacting the cell with an rAAV of the disclosure. In another aspect, the disclosure provides a method of increasing eEF1A2 activity in a subject, comprising administering to an rAAV of the disclosure. In some embodiments, the cell and/or subject is deficient in eEF1A2 expression levels and/or activity and/or comprises a loss-of-function mutation in eEF1A2. The cell may be a neuron, e.g. a dopaminergic neuron.

In some embodiments, the method promotes survival of neurons in cell culture and/or in vivo.

### Methods of Treatment

In another aspect, the disclosure provides a method of treating a disease or disorder in a subject in need thereof, comprising administering to the subject an effective amount of an rAAV virion of the disclosure. In some embodiments, the disease or disorder is a neurological disease or disorder. In some embodiments, the subject suffers from a genetic disruption in eEF1A2 expression or function. In some embodiments, the disease or disorder is an eEF1A2 deficiency and/or an eEF1A2-related neurological disease (OMIM #617309, 616393, 616409) phenotypic spectrum, such as intellectual disability, mental retardation, epileptic encephalopathy and autism spectrum disorder.

The AAV-mediated delivery of eEF1A2 protein to the CNS may increase life span, prevent neuronal degeneration, prevent or attenuate neurobehavioral deficits, degenerative epileptic-dyskinetic encephalopathy, epilepsy, and dystonia.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid to prevent or to reduce an immune response to administration of a rAAV described herein.

A therapeutically effective amount of the rAAV vector, e.g. for intracerebroventricular (ICV) or intra-cisterna magna (ICM) injection, is a dose of rAAV ranging from about 1e12 vg/kg to about 5e12 vg/kg, or about 1e13 vg/kg to about 5e13 vg/kg, or about 1e14 vg/kg to about 5e14 vg/kg, or about 1e15 vg/kg to about 5e15 vg/kg, by brain weight. Or intravenous delivery dose range from 1213-1e14vg/kg by body weight. The invention also comprises compositions comprising these ranges of rAAV vector.

For example, in particular embodiments, a therapeutically effective amount of rAAV vector is a dose of about 1e10 vg, about 2e10 vg, about 3e10 vg, about 4e10 vg, about 5e10 vg, about 6e10 vg, about 7e10 vg, about 8e10 vg, about 9e10 vg, about 1e12 vg, about 2e12 vg, about 3e12 vg, about 4e12 vg, or about 5e12 vg. The invention also comprises compositions comprising these doses of rAAV vector.

In some embodiments, for example where ICV injection is performed, a therapeutically effective amount of rAAV vector is a dose in the range of 1e10vg/hemisphere to 1e13 vg/hemisphere, or about 1e10 vg/hemisphere, about 1e11 vg/hemisphere, about 1e12 vg/hemisphere, or about 1e13 vg/hemisphere. In some embodiments, for example where ICM injection is performed, a therapeutically effective amount of rAAV vector is a dose in the range of 1e10 vg total to 1e14 vg total, or about 1e10 vg total, about 1e11 vg total, about 1e12 vg total, about 1e13 vg total, or about 1e14 vg total.

In some embodiments, the therapeutic composition comprises more than about 1e9, 1e10, or 1e11 genomes of the rAAV vector per volume of therapeutic composition injected. In embodiments cases, the therapeutic composition comprises more than approximately 1e10, 1e11, 1e12, or 1e13 genomes of the rAAV vector per mL. In certain embodiments, the therapeutic composition comprises less than about 1e14, 1e13 or 1e12 genomes of the rAAV vector per mL.

Evidence of functional improvement, clinical benefit or efficacy in patients may be assessed by the analysis of surrogate markers of reduction in seizure frequency (myoclonic and generalized tonic clonic seizures), brain growth and body growth using UK-WHO paediatric head circumference, height and weight percentile charts. Measures in cognition, motor, speech and language function using standard disease rating scales, such as Childhood seizure inventory and medication log. Cognitive and Developmental Assessments including the Peabody Developmental Motor Scales 2^{nd} edition (PDMS-2) and Bayley Scales of Infant Development, 3^{rd} edition applied as appropriate to level of child’s disability. Gross motor function measure (GFMF-88), Pediatric Evaluation of Disability Inventory (PEDI). These or similar scales, as well as patient-reported outcomes on quality of life such as Caregiver Global Impression of Change in Seizure Duration (CGICSD) on a 3-point scale (decrease, no change, or increase in average duration), Pediatric Quality of Life Inventory (PedsQL™) and Vineland Adaptive Behavior Scales-2nd may demonstrate improvements in components of the disease. Baseline and post treatment Brain magnetic resonance imaging may show improvements in myelination and brain volume. Cardiac defects have been observed in patients with autosomal dominant EEF1A2-related neurodevelopmental disorder, including cardiomyopathy, aortic defects and ventricular septal defect (Kaneko et al., 2021, Carvill et al., 2020; McLachlan et al., 2019). A homozygous variant in EEF1A2 was identified in single kindred with global developmental delay, epilepsy, failure to thrive, dilated cardiomyopathy and premature death (Cao et al., 2017). Measures of cardiac status maybe monitored through baseline electrocardiogram and echocardiogram.

Clinical benefit could be observed as increase in life-span, meeting normal neurodevelopmental milestones, decreases in frequency or magnitude of epileptic seizure activity (including myoclonic, clonic, generalized tonic-clonic and/or epileptic spasm), improvement in, or lack of developing hypotonia or movement disorders such as choreoathetosis, dystonia, and/or ataxia. Evidence of neuroprotective and/or neurorestorative effects may be evident on magnetic resonance imaging (MRI) by characterizing degree of myelination across development, thickness of corpus callosum, and degree of cortical and/or cerebellar atrophy. Beneficial changes in electroencephalogram (EEG) activity would be evident by decreases in multifocal discharge and/or generalized spike activity.

In some embodiments, for example where intravenous administration is performed, a therapeutically effective mount of rAAV vector is a dose in the range of about 1e12 vg/kg to 1e14 vg/kg by total body weight of the subject. For example, in particular embodiments, a therapeutically effective amount of rAAV vector is a dose of about 1e12 vg/kg, about 2e12 vg/kg, about 3e12 vg/kg, about 4e12 vg/kg, about 5e12 vg/kg, about 6e12 vg/kg, about 7e12 vg/kg, about 8e12 vg/kg, about 9e12 vg/kg, about 1e13 vg/kg, about 2e13 vg/kg, about 3e13 vg/kg, about 4e13 vg/kg, about 5e13 vg/kg, about 6e13 vg/kg, about 7e13 vg/kg, about 8e13 vg/kg, about 9e13 vg/kg, or about 1e14 vg. Evidence of cardiac benefit may include stable cardiac function on echocardiogram.

### Administration of Compositions

Administration of an effective dose of the compositions may be by routes standard in the art including, but not limited to, systemic, local, direct injection, intravenous, cerebral, cerebrospinal, intrathecal, intracisternal, intraputaminal, intrahippocampal, intra-striatal (putamen and/or caudate), intracortical, or intra-cerebroventricular administration. In some cases, administration comprises intravenous, cerebral, cerebrospinal, intrathecal, intracisternal, intraputaminal, intrahippocampal, intra-striatal (putamen and/or caudate), or intra-cerebroventricular injection. Administration may be performed by intrathecal injection with or without Trendelenberg tilting.

In some embodiments, the disclosure provides for local administration and systemic administration of an effective dose of rAAV and compositions of the invention. For example, systemic administration may be administration into the circulatory system so that the entire body is affected. Systemic administration includes parental administration through injection, infusion or implantation.

In particular, administration of rAAV of the present invention may be accomplished by using any physical method that will transport the rAAV recombinant vector into the target tissue of an animal. Administration includes, but is not limited to, injection into the central nervous system (CNS) or cerebrospinal fluid (CSF) and/or directly into the brain.

In some embodiments, the methods of the disclosure comprise intracerebroventricular, intracisternal magna, intrathecal, or intraparenchymal delivery. Infusion may be performed using specialized cannula, catheter, syringe/needle using an infusion pump. Optionally, targeting of the injection site may be accomplished with MRI-guided imaging. Administration may comprise delivery of an effective amount of the rAAV virion, or a pharmaceutical composition comprising the rAAV virion, to the CNS. These may be achieved, e.g., via unilateral intraventricular injection, bilateral intraventricular injection, intracisternal magna infusion with Trendelenburg tilting procedure, or intracisternal magna infusion without Trendelenburg tilting procedure, intrathecal infusion with Trendelenburg tilting procedure, or intrathecal infusion without Trendelenburg tilting procedure. The compositions of the disclosure may further be administered intravenously.

Direct delivery to the CNS could involve targeting the intraventricular space, either unilaterally or bilaterally, specific neuronal regions or more general brain regions containing neuronal targets. Individual patient intraventricular space, brain region and/or neuronal target(s) selection and subsequent intraoperative delivery of AAV could by accomplished using a number of imaging techniques (MRI, CT, CT combined with MRI merging) and employing any number of software planning programs (e.g., Stealth System, Clearpoint Neuronavigation System, Brainlab, Neuroinspire etc). Intraventricular psace or brain region targeting and delivery could involve us of standard stereotactic frames (Leksell, CRW) or using frameless approaches with or without intraoperative MRI. Actual delivery of AAV may be by injection through needle or cannulae with or without inner lumen lined with material to prevent adsorption of AAV vector (e.g. Smartflow cannulae, MRI Interventions cannulae). Delivery device interfaces with syringes and automated infusion or micr30oinfusion pumps with preprogrammed infusion rates and volumes. The syringe/needle combination or just the needle may be interfaced directly with the stereotactic frame. Infusion may include constant flow rate or varying rates with convection enhanced delivery.

### EXAMPLES

### Example 1: Promoter Selection

Biodistribution studies in wildtype neonatal mice were performed to select a promoter to restore expression of eEF1A2 in neurons. The human synapsin (hSYN) promoter showed superior selectivity for the nervous system and strong neuronal expression compared to all other candidate promoters, as shown in Table 1. Surprisingly, the hSYN promoter showed greater neuronal selectivity than eSYN and other promoters tested.

TABLE 6

| | |
|---|---|
| Promoter | Brain cellular tropism of eEF1A2 transgene |
| CMV | Neuronal and glial |
| EF1a | Strong neuronal |
| Enhanced Synapsin (eSYN) | Neuronal and glial |
| Human Synapsin (hSYN) | Strong neuronal |

### Example 2: Aav9 Gene Therapy Rescue of an Eef1a2 Knockout Mouse Model

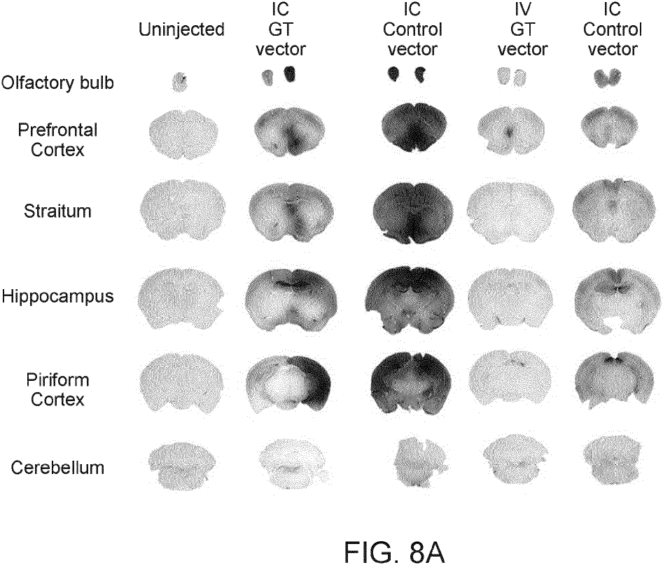

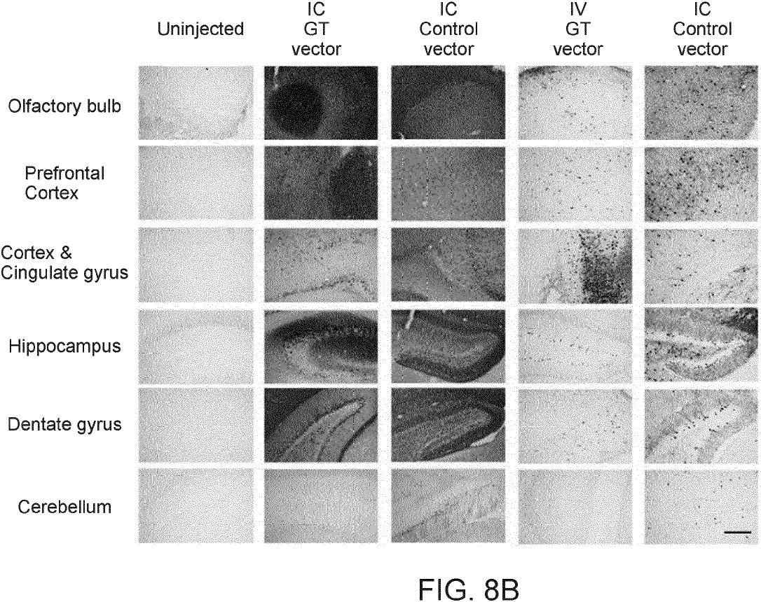

We have developed a new treatment approach for subjects (e.g., children) affected by mutations in the EEF1A2 gene. Eukaryotic translation elongation factor 1 alpha 2 (eEF1A2) is essential for the delivery of aminoacyl transfer RNA to the ribosome for protein synthesis. Mutations in the EEF1A2 gene have been associated with severe intellectual disability, autism and epilepsy. There are currently no effective treatments. An EEF1A2 knockout mouse model (wasted mice) has been well-characterized. The wasted (wst/wst) mice exhibit gait disturbances and tremor after weaning, followed by paralysis and motor neuron degeneration by 23 days of age. Using this mouse model, the inventors tested whether the function of the protein could be restored with gene therapy. We designed an adeno-associated virus 9 (AAV9) using a pan neuronal promoter, human Synapsin, to drive expression of the human EEF1A2 cDNA (hSyn-eEF1A2). An eGFP marker gene was included to track expression of the construct in vivo. Immunofluorescence (FIG. 7) revealed neuronal targeting after neonatal IC or IV injection of AAV9-hSyn-eEF1A2-T2A-eGFP. Immunohistochemical staining (FIG. 8) confirmed widespread transgene expression in the CNS after both routes of administration from a single injection of a rAAV (for both eEF1A2-2A-eGFP or eGFP marker alone).

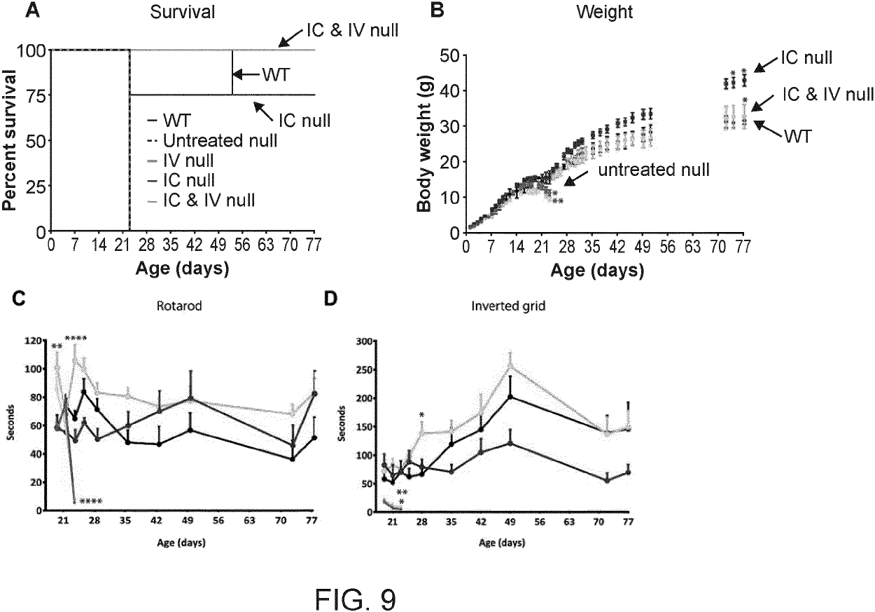

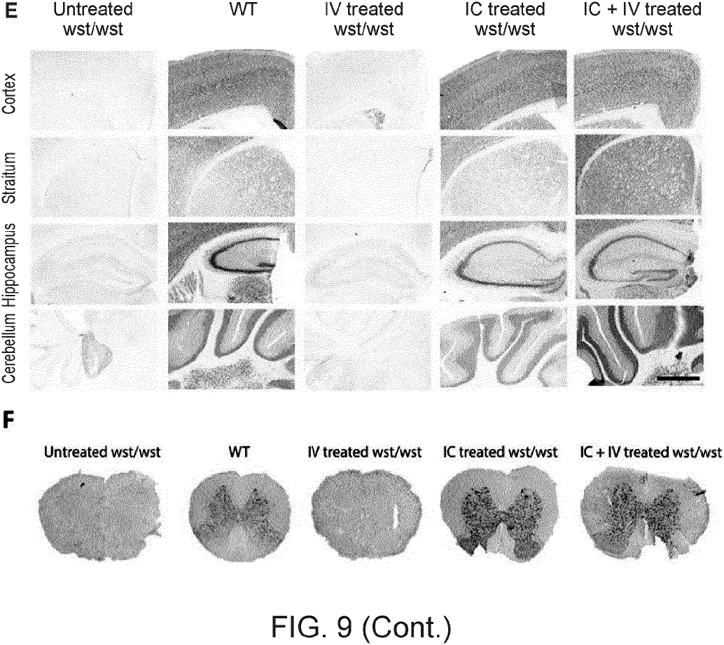

The gene therapy vector proved effective in treating wasted (wst/wst) mice. Eef1a2^{-/-} knockout mice (wst/wst) mostly survived (¾) when injected IC and all survived when injected both IC and IV (FIG. 9A). Untreated mice died by P23. IC or IC/IV mice similarly showed no weight loss compared to WT mice, whereas untreated control mice exhibit weight loss leading to death by P23 (FIG. 9B). Rotarod and inverted grid analysis demonstrated no decline in performance in the treated (FIG. 9C and FIG. 9D). The results were significant by both two-way ANOVA and Dunnett’s multiple comparison tests.

eEF1A2 expression was observed throughout the brain in wild-type, IC and combined treatment (FIG. 9E and FIG. 9F). eEF1A2 expression was present in spinal cord tissue of wild-type, IC and combined treated groups. However, expression was absent in the untreated wasted group and IV treated groups (F).

### Example 3: AAV9 Gene THerapy Rescue of EEF1A2^{D252H} or EEFlA2^{G70S} or EEF1A2^{E122K} Mouse Models

Efficacy of vector designs shown in FIGS. 2-5 and FIG. 6, as well as various codon-optimizations, are compared to identify the vectors that have superior efficacy. Experiments are performed in mouse models that recapitulate three mutations found in humans (D252H, G70S and or E122K) and/or a mouse model with a severe neurodegenerative phenotype (Del.22.ex3). Experiments are performed in both neonatal mice and at later stages of development through adult to confirm AAV vectors encoding eEF1A2 can rescue survival, weight loss, and behavioral phenotypes. Assessment of beneficial effects on neurobehavioral tests includes performance on a rotating cylinder (rotarod), ability to cling to a suspended wire surface (wire hang test), foot fault test, inverted grid behavior, grip strength, and behavior in an open field including observations of normal exploratory activity or abnormal behavior (e.g. “twitching behavior”). A collective neuroscore on a battery of tests may be obtained to assess neurobehavioral function of AAV9-eEF1A2 injected mice relative to nontreated controls, that includes analysis of hindlimb clasping, gait, kyphosis and ability to walk along a ledge. Alterations in frequency of seizures and electroencephalography (EEG) in the Del.22.ex3 (or other mouse strains with abnormal EEG) mouse that have enhanced life span reveal beneficial effects in mitigating abnormal electrical activity in CNS. Biochemical and histological analyses confirm superior efficacy of vector designs on eEF1A2 expression levels and distribution within CNS. Tissue analyses includes detection of mRNA, DNA, vector copy number, and transgene protein expression in fresh tissue by Western blot and ELISA and in fixed sections of CNS by immunolabeling.

### Example 4: Effects of Aav9-Mediated Delivery of Human Eef1a2 Protein to the Central Nervous SYystem in the WST/WST Mouse Model of EEF1A2 Insufficiency

A leading model for eEF1A2 related disorders is the Wasted mouse model, in which spontaneous deletion of the first exon and all promoter elements of the EEf1A2 gene results in eEF1A2 null (wst/wst). In untreated animals, 31% of wst/wst mice die between P20-22 and the surviving wst/wst mice show attenuation of weight, tremors followed by weight loss, with the remainder all dying by day 24. The untreated animals also may exhibit impaired grip strength and impaired rota rod performance with animals surviving the longest developing tremors, progressive paralysis and weight loss. The animals in our wst/wst colony appear more severe with more acute decline and earlier death. We have observed the impaired grip strength through inverted grid latency to fall at P23. With the wst/wst animals only surviving to P24, we have not observed a deterioration in rota rod performance to significantly distinguish wst/wst from wildtype animals.

### Materials and Methods

### Animal Welfare

All animal experiments were performed in compliance with UK Home Office and the Animal (Scientific Procedures) Act of 1986, and within the guidelines of University College London ethical review committee. The wst/wst eEF1A2 null mouse model used in this study has been described previously (Chambers D et al. PNAS 95:4463-8 (1998).). Heterozygous mice were time mated to generate mixed genotype litters. Pups were genotyped at P0 using primers (Primers EEF1A2 Mut F 5′ ACCAGTGGTTTCACCTGCTC 3′, EEF1A2 Common R 5′ CACTGTGGGGGCTCTGGTTT 3′, EEF1A2 WT F 5′ CAGAGCTTCACTCAGTCTG 3′).

Administration of the AAV9-eEF1A2 vectors or control articles were performed by bilateral intracerebroventricular injection to neonatal homozygous wst/wst or WT littermate pups at P0 and animals were followed to humane endpoint (weight loss ≥15%) or timed sacrifice point P60. The intracerebroventricular injections were directed to the lateral ventricle of P0-1 mice as described previously (Newbery HJ, et al. J Neuropathol Exp Neurol 64:295-303 (2005)). A 33-gauge needle (Hamilton) was inserted perpendicularly at the injection site to a depth of 3 mm and 5 µl of vector was administered over 5 seconds into the lateral ventricle. The pup was returned to dam promptly. Group sizes were 6 for gene therapy treated wst/wst mice with 14-16 control littermates across 7 litters.

### Behavioural Studies

Mice were weighed regularly and assessed for changes in general well-being and meeting humane endpoint. Behavioural testing Rotarod, and inverted Grid test were performed at P23. All behavioural testing were performed by researchers blinded to animal treatment group. Mice were placed on the rotarod (Harvard Apparatus®) under continuous acceleration from 4-40 r.p.m. for maximum 5 minutes. The time at which the mice fell off the rod was recorded with 3 trials (latency to fall) for each animal on each day of testing. The Inverted Grid test involved placing the mouse on a stainless-steel grid (41 × 25 cm) which was placed over a 30 cm elevated plastic transparent box. The latency to fall from the inverted grid was recorded, with a maximum 5 minutes. The Inverted Grid test was repeated 3 times per mouse on each day of testing.

### Histological and Immunohistochemical Analyses of Mouse Tissues

Mice were culled by terminal transcardial perfusion using PBS. Collected tissues (brain and visceral organs) were halved to allow for different processing techniques. Brains used for immunohistochemistry were post-fixed in 4% PFA for 48 hours and transferred into 30% sucrose solution for cryoprotection at 4° C. until sectioning. Brains were mounted on a freezing microtome (ThermoFisher® HM430) at 40 µm thickness in either coronal planes. Free-floating immunohistochemistry-based analyses was performed with brain sections selected at 240 µm intervals for whole-brain immunohistochemistry. Briefly, free-floating sections were blocked in 15% normal goat serum (Vector Laboratories®)- tris buffered saline with 0.1% triton-X (TBS-T) (Sigma®) for 1 hour at room temperature and incubated in primary antibodies Rabbit eEF1A2 (Proteintech®) in 10% normal goat serum-TBS-T overnight at 4° C. The following day sections are incubated with the respectively species-specific secondary antibodies (Vector Laboratories®) for 1 hour at room temperature, washed in TBS followed by incubation with Vectastain avidin-biotin solution (Vector Laboratories®). The reaction visualized with 3,3′-Diaminobenzidine (DAB) (Sigma®). DAB reaction was stopped using ice cold 1× TBS and sections washed before mounting on double coated gelatinized glass slides. The mounted sections were air dried and dehydrated in 100% ethanol for 10 minutes and dehydration solution (Histoclear™, National Diagnostics®) for 30 minutes prior to being covered with mountant (DPX, VWR International®) for coverslipping.

For immunofluorescence brain sections were blocked in 15% goat serum for 30 minutes and then incubated with primary antibodies (Rabbit eEF1A2 1:1000 Proteintech® and Mouse NeuN 1:1000 Milipore) diluted in 10% normal goat serum TBS-T 0.3% overnight at 4° C. The sections were washed in 1×TBS and incubated for 2 hours with the respectively species-specific secondary antibodies labelled with Alexa 488 and Alexa 594 (all from Invitrogen®) diluted in 10% normal goat serum at room temperature. NV4ei were stained with DAPI (Sigma Aldrich®) for 2 minutes. The brain sections were mounted onto double coated slides and coverslipped using Fluromount G™ (Thermofisher Scientific®).

Light microscopy and fluorescence imaging were carried out using a Leica DM 4000 linked to Leica DFC420 camera system. Confocal images were captured using a Leica TCS SP5 AOBS confocal microscope. Images were analyzed with Image J software (National Institutes of Health).

### Immunoblotting

Proteins were extracted from mouse brain tissue in ice-cold 0.32 M sucrose supplemented with protease inhibitor (Roche®) using Qiagen® tissue lyser and centrifuged at 4 degrees for 15 minutes. Protein concentration was measured with Pierce BCA Protein Assay kit (Thermo Scientific®): 10 µg of protein was denatured with Laemmli buffer (Bio-Rad Laboratories®) with dithiothreitol (DTT). Proteins were separated with Mini-PROTEAN TGX™ Stain Free Gels (Bio-Rad Laboratories®) and transferred to a Trans-Blot Turbo Transfer membrane (Bio-Rad Laboratories®). After blocking in Biorad® blocking buffer for 1 hour at room temperature, membranes were incubated with primary antibodies rabbit eEF1A2 (Proteintech®, 1:1000) and mouse GAPDH (Ab Cam®, 1:10,000) at 4° C. overnight. Membranes were then incubated with the secondary StarBright™ Blue 520 Goat Anti-Rabbit IgG (1:3000) and StarBright™ Blue 700 Goat Anti-Mouse IgG, (1:3000). Immunoreactive proteins were visualized with Chemidoc MP (Bio-Rad Laboratories®). Samples loaded n=4-5 biological replicates

### qRT-PCR mRNA Transcript Expression Analysis

RNA was extracted from brain homogenates (forebrain, cortex n=4-5 biological replicates per group) extracted with RNeasy™ mini kit (Qiagen®) following the manufacturer’s instructions and quantified on Omega Fluostar™. Contaminating DNA was removed from total RNA (1 µg) using the DNAse I purification kit (NEB®), before performing reverse transcription with High-Capacity cDNA Reverse Transcription Kit (Applied Bioscience®). Then 10 ng of DNA or synthesized cDNA was used to perform the multiplex hEEF1A2 and mGAPDH RT-qPCR (eEF1A2_Fwd1: ATCGTGGGCGTGAACAAA, eEF1A2_Rev1:GGTTGTAGCCGATCTTCTTGAT, eEF1A2_Probe: ATCGTCAAGGAAGTCAGCGCCTAC and mouse GAPDH For: ACGGCAAATTCAACGGCAC, Rev: TAGTGGGGTCTCGCTCCTGG, Probe: TTGTCATCAACGGGAAGCCCATCA with Luna Taqman™ mastermix (NEB®) in Quantstudio™ Real-Time PCR System (Applied Biosystems®). GAPDH was used as endogenous controls and relative fold change calculated.

### Statistical Analysis

Statistical analysis tailored to each experiment was performed using GraphPad Prism™ version 8. In vivo experimental design and sample sizes were designed using NC3Rs guidance and power calculation. For most analyses of animal experiments, one-way or two-way ANOVA was performed with either Bonferroni or Tukey’s multiple comparison.

### Results

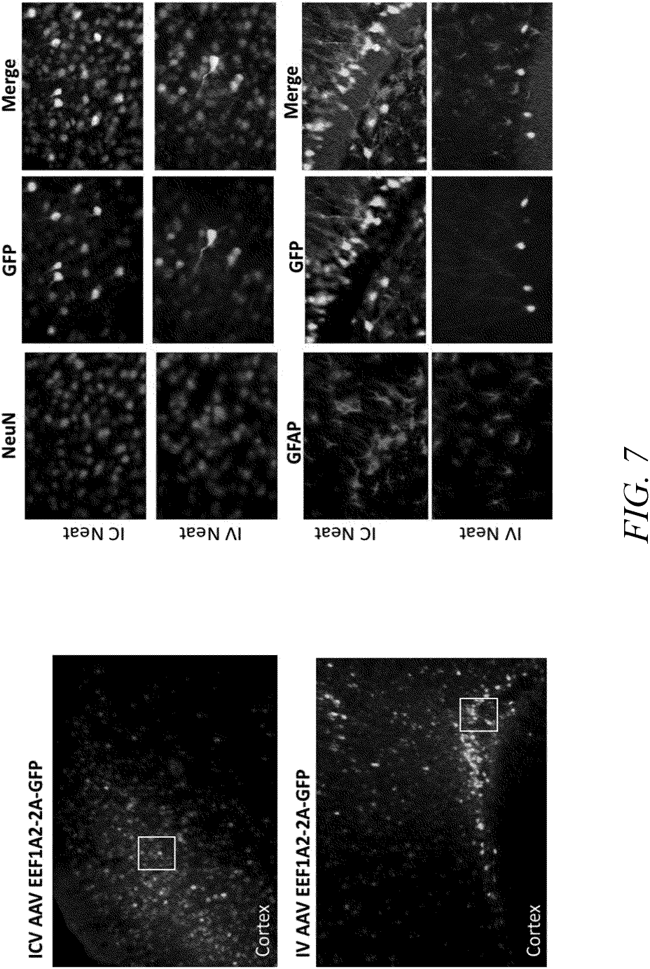

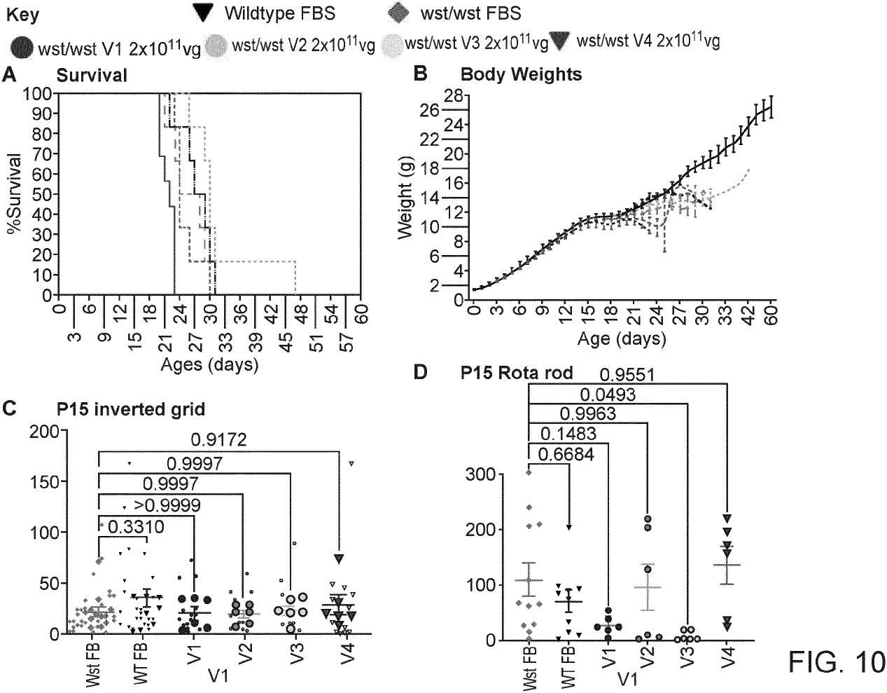

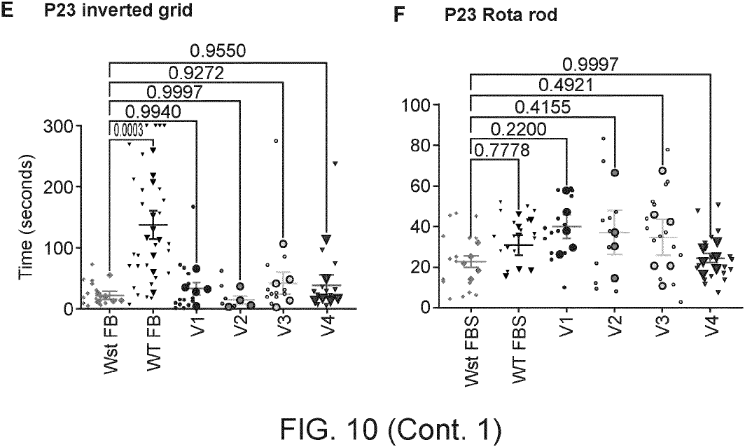

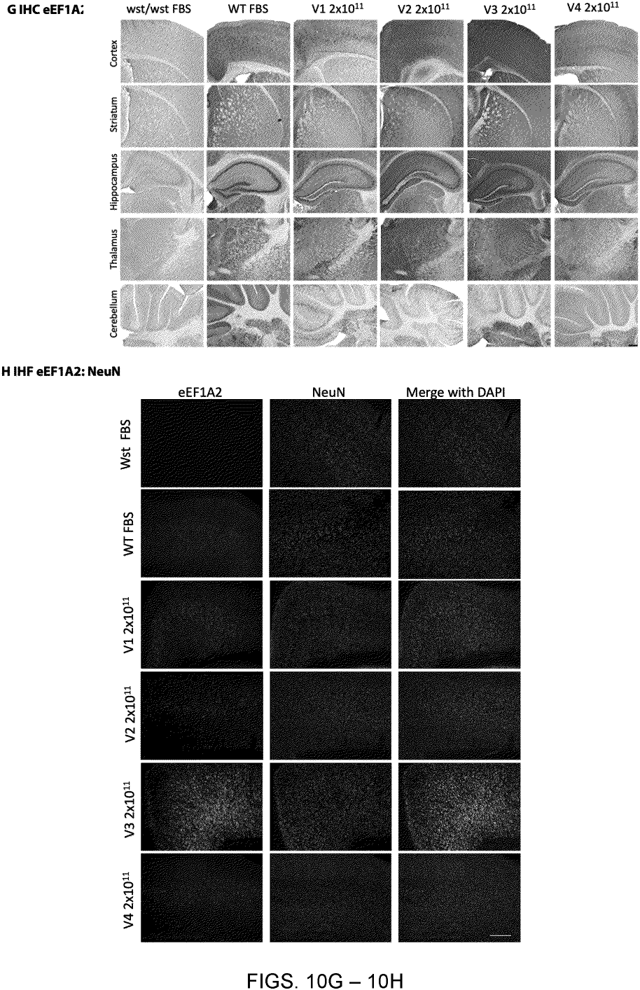

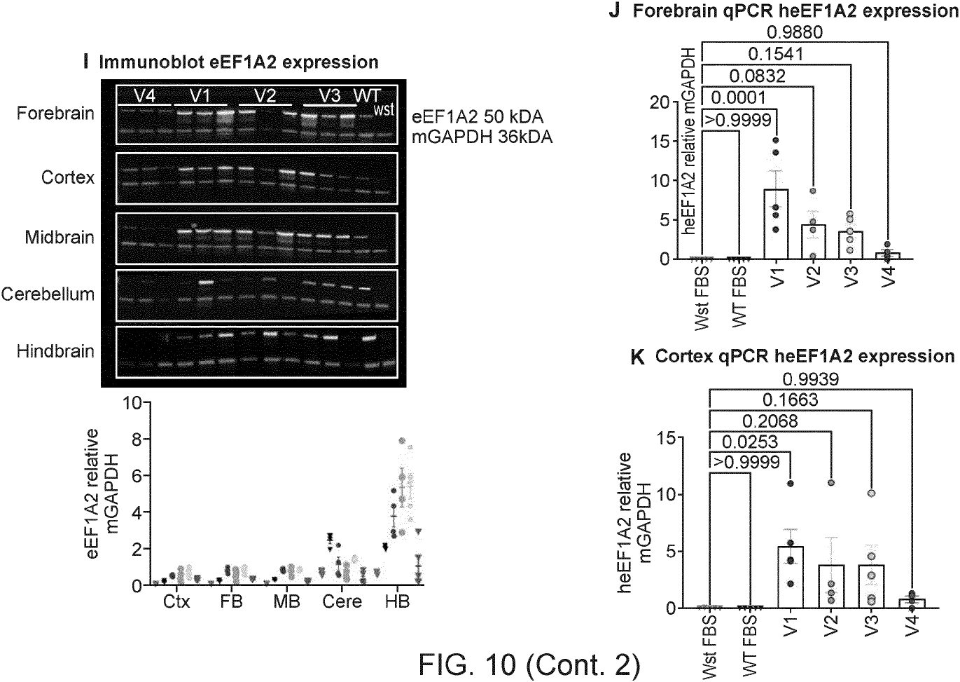

FIGS. 10A-10K shows comparisons of AAV9 vectors comprising the vector genomes shown in FIG. 2 (“V1”; SEQ ID NO: 55), FIG. 3 (“V2”; SEQ ID NO: 56), FIG. 4 (“V3”; SEQ ID NO: 57) and FIG. 6 (“V4”; SEQ ID NO: 58) administered at 2e10¹¹ vg/animal.

FIG. 10A Kaplan-Meier survival plot of FBS treated wildtype, wst/wst, intracerebroventricular treated wst/wst animals with V1, V2,V3 and V4 gene therapy treated (2 × 10¹¹ vg/pup, all gene therapy treated groups n=6, wildtype FBS n = 14, wst/wst FBS= 17. All gene therapy treated animals showed significant extension of life (mean extension V1=4.4 days,p=0.001, V2=3.8 days p=0.0014, V3 10.7 days p<0.0001, V4 =4.4 days p=0.001 Logrank Mantel-Cox test). FIG. 10B Weights of mice (Data means ± S.E.M.) animals were weighed daily until postnatal age 35 and weekly thereafter until timed sacrifice point P60 or humane endpoint 15% weight loss. FIG. 10C, FIG. 10D, FIG. 10E, and FIG. 10F Muscle strength assessment by inverted grid tests and rota rod (n=4-7 per group, each animal tested in triplicate at 15 and 23 days old). No significant difference was observed with these tests between wst/wst and wildtype FBS control groups on rota rod. Significant reduced grip strength in wst/wst FBS animals was observed at P23 compared to WT and no effect of gene therapy vectors was seen on these behavioural outcomes (Data means ± S.E.M., two-way ANOVA). FIG. 10G Representative immunostaining for eEF1A2 throughout the brain by free floating immunohistochemistry with wildtype FBS as physiological reference (n=4-5 per group, scale bar 250 µm) All wst/wst animals injected with V1-4 express eEF1A2 throughout the brain with darkest staining and highest expression observed with V3. FIG. 10H Representative immunohistofluorescence in cortical brain regions for neurons (NeuN marker) co-expressing eEF1A2 (n=4.5 per group, 200 µm). All show neuronal expression profile with higher expression compared with wildtype. The highest expression observed through fluorescence intensity, was with V3 showing expression through all cortical layers. All images were captured a constant imaging settings. More discrete expression in cortical layers 4 and 5 observed with V2 and V4. FIG. 10I Representative immunoblot for eEF1A2 in brain with quantification showing eEF1A2 expression throughout the brain achieved with all gene therapy vectors with higher expression in midbrain, cerebellar and hindbrain regions compared to V4 vector (Data means ± S.E.M, two-way ANOVA). FIG. 10J qPCR for human eEF1A2 transcript expression in forebrain showing highest mRNA expression with V1 vector (Data means ± S.E.M, two-way ANOVA). FIG. 10K qPCR for human eEF1A2 cortex expression in forebrain showing highest mRNA expression with V1 vector (Data means ± S.E.M, two-way ANOVA).

### Conclusions

These experiments demonstrate that all four vectors are capable of restoring expression of eEF1A2 in a mouse model having homozygous null mutations in the EEF1A2 gene (termed wst in mice). Surprisingly, vectors V1, V2, and V3 are able to effectively and significantly extend survival in wst/wst mice beyond P23. (FIG. 10A). Moreover, the experiment unexpectedly demonstrates that expression of the eEF1A2 transgene in the brain, specifically the forebrain (FIG. 10J) and the cortex (FIG. 10K), is greater in V3 than in V1, V2 or V4. Without being bound by theory, it appears that contrary to expectations including the 5′ UTR (SEQ ID NO: 34) and/or 3′ UTR (SEQ ID NO: 48) therapeutically increases gene expression, extending survival.

### Example 5: Effects of Aav9-Mediated Delivery of Human Eef1a2 Protein to The Central Nervous system in the D252H Mouse Model of EEF1A2 Disease

Heterozygous de novo mutations in EEF1A2, encoding the tissue-specific translation elongation factor eEF1A2, have been shown to cause neurodevelopmental disorders including often severe epilepsy and intellectual disability. There are approximately 50 different missense mutations identified but no obvious loss of function mutations, though large heterozygous deletions are known to be compatible with life. A knock-in eEF1A2 mouse model harboring a disease causing missense D252H mutation are more severely affected than null homozygotes on the same genetic background showing attenuation of weight, increasing neuroscore, and death by P23. Mice that are heterozygous for the missense mutation show no behavioural abnormalities but do have sex-specific deficits in body mass and motor function with transient impaired grip strength. The phenotyping of this D252H novel mouse alongside del22ex3 null mouse model supports D252H mutation results in a gain of function (Davies, Faith CJ, et al. Human Molecular Genetics (2020)). This Example describes gene therapy studies in both heterozygous and homozygous D252H mice.

### Materials and Methods

Survival and weight of the treated and untreated mice was monitored over the course of development. Behavioral tests were also performed between P18-24 days of age.

### Animal Welfare

All animal experiments were performed in compliance with UK Home Office and the Animal (Scientific Procedures) Act of 1986, and within the guidelines of University College London ethical review committee. Heterozygous D252H eEF1A2 knock-in mice were time mated to generate mixed genotype litters. Pups were genotyped at P0 using primers 5″-3″ AGGCTACCCCTTAGGCAGGT, TGAACAAATGGTAGGTGGGAGG. After PCR amplification, samples were subjected to restriction digest by Hin1II (Thermo Fisher).

### Administration of Test and Control Articles

Administration of 5µL of V3 vector or Formulation Buffer (FB) article was achieved by a unilateral intracerebroventricular injection of a dosage of 1.8×10¹¹vg/pup to neonatal homozygous knock-in mice (D252H-/-). Test or FB Control articles were administered to wildtype and heterozygous D252H through a 33-gauge Hamilton needle (Fisher Scientific®, Loughborough, UK) using injection site coordinates delineated by Kim et al. Kim, J. Y. et al. J Vis Exp 91:51863 (2014). The lambdoid suture is identifiable in neonatal pups and the intended injection site is ⅖ths from lambdoid suture to the eye located approximately 0.8 mm- 1 mm lateral from sagittal suture, halfway between lambda and bregma. After injections pups were returned to their dams.

### Body Weight

Individual animal body weight was collected from P1-P32, then weekly thereafter. Weight loss of ≥15% will reach humane endpoint criteria for euthanasia.

### Behavioral Testing

All behavioral testing assays were performed by researchers blinded to animal treatment group. Rota rod Test: Rotarod training/testing from P18-24 was performed. Mice were placed on the rotarod (Harvard Apparatus) under continuous acceleration from 4-40 r.p.m. for a maximum of 2 minutes. The time at which the mice fell off the rod was recorded with 3 trials (latency to fall) for each animal on each day of testing. Inverted Grid Test: Inverted Grid testing involves placing the mouse on a stainless-steel grid (41 × 25 cm) that is placed over a 30 cm elevated plastic transparent box. The latency to fall from the inverted grid is recorded, with a maximum of 2 minutes. The Inverted Grid test is repeated 3 times per mouse on each day of testing.

### Results

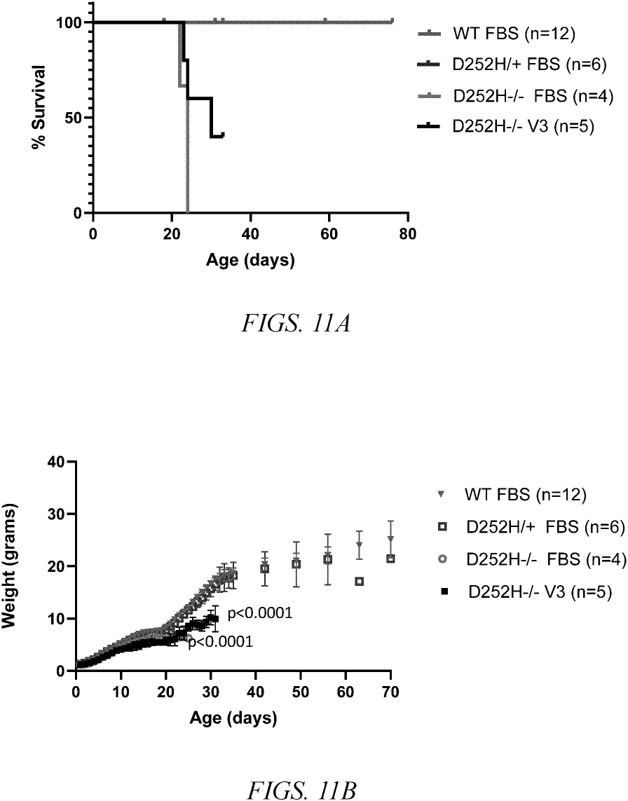

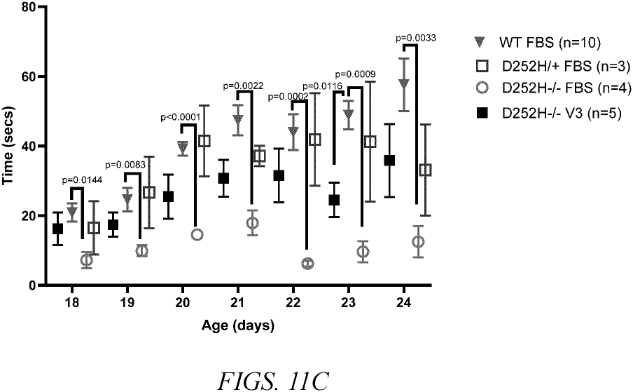

FIGS. 11A-11C show the phenotype of untreated D252H-/- mice. FIG. 11A: Kaplan-Meier survival plot of FBS treated wildtype, knock-outs (D252H-/-), intracerebroventricular V3 gene therapy treated D252H-/- (2 × 10¹¹ vg/pup, V3 treated = 2, V4 treated=1, wildtype FBS n = 5, D252H-/- FBS= 3. FIG. 11B Weights of mice (Data means ± S.E.M.). FIG. 11C Motor assessment by rotarod (Data means ± S.E.M., two-way ANOVA, and Dunnett’s multiple comparison).

### Conclusions

These experiments demonstrate that AAV9-mediated expression of eEF1A2 in D252H-/- mice with V3 can increase survival compared to untreated D252H-/- controls. Additionally, although rota rod performance is consistently poor in D252H -/- homozygous mice, a trend for improvement is consistently observed across time in D252H-/- V3 treated mice. In an a priori defined longer-term safety/tolerability study, overexpression of eEF1A2 following intracerebroventricular administration of V3 in D252H/+ heterozygous mice does not appear to have deleterious effects on survival or functional outcome measures. Ongoing analyses in both homozygous and heterozygous D252H mice will further characterize the longer-term effects of AAV9-mediated eEF1A2 overexpression in this model of eEF1A2 deficiency.

### Example 6: Effects of Aav9-Mediated Delivery of Human Eef1a2 Protein to The Central Nervous sysem in the DEL22EX3 Mouse Model of EEF1A2 Disease

A CRISPR/Cas9 generated Del.22.ex.3 eEF1A2 mouse model was generated to knock out eEF1A2 expression (Davies, Faith CJ, et al. Human Molecular Genetics 2020). A 22 base pair deletion within exon 3 of Eef1a2 that was generated from CRISPR/Cas9 mutagenesis resulted in a null mutation. These Del22ex3 mice present a severe phenotype such that mice do not survive much longer after the onset of disease (~ 21-25 days) suffering from early onset motor neuron degeneration with paralysis with additional clinically relative symptoms of fatal epileptic seizures. This Example describes gene therapy studies in homozygous Del22ex3 eEF1A2 null mice.

### Materials and Methods

Survival and body weight of the treated and untreated mice was monitored over the course of development. Behavioral tests were also performed between the critical window of P21-25 days of age.

### Animal Welfare

All animal experiments were performed in compliance with UK Home Office and the Animal (Scientific Procedures) Act of 1986, and within the guidelines of University College London ethical review committee. Heterozygous Del22ex3 mice were time mated to generate mixed genotype litters. Pups were genotyped at P0 using primers 5″-3″ 5′-TGAGTTGTGCCTCTACCCTT-3′ and 5′-TACAGGCACATCCCAGGTGT-3′

### Administration of Test and Control Articles

Intracerebroventricular injections of 10µL (5µL in each hemisphere, bilaterally) of V3 vector or Formulation Buffer (FBS) were administered to neonatal homozygous Del22ex3 mice (Del22ex3) pups at a dose of 2×10¹¹vg/pup (V3 high dose) or 2× 10¹⁰vg/pup (V3 Low dose). Formulation buffer solution (FBS, 5µL bilaterally) as a control was administered to wildtype and homozygous Del22ex3 mice through a 33-gauge Hamilton needle (Fisher Scientific®, Loughborough, UK) using injection site coordinates delineated by Kim et al. Kim, J. Y. et al. J Vis Exp 91:51863 (2014). The lambdoid suture is identifiable in neonatal pups and the intended injection site is ⅖ths from lambdoid suture to the eye located approximately 0.8 mm- 1 mm lateral from sagittal suture, halfway between lambda and bregma. After injections pups were returned to their dams.

### Body Weight

Individual animal body weight was collected daily from P1-P30, then weekly thereafter. Weight loss of ≥15% was utilized as humane endpoint criteria for euthanasia.

### Behavioral Testing

Rotarod training and testing occurred on days P21-25 (P21 is considered training day). All behavioral assays were performed by researchers blinded to animal treatment group. Mice were placed on the rotarod (Harvard Apparatus) under continuous acceleration from 4-40 r.p.m. for a maximum of 2 minutes. The time at which the mice fell off the rod was recorded with 3 trials (latency to fall) for each animal on each day of testing.

Limb muscle strength was measured between P21-25 using a grip strength meter (Bioseb®) (P21 is considered training day). Grip strength from all four limbs or front limbs was measured in triplicate with a 1-minute break in between each test to allow the mouse to rest. For each test the mouse was held by the base of the tail and lowered onto the grid until it gripped with either the front paws or all four paws.

### Neuroscore

Ledge test: Each mouse was placed on the ledge of an empty cage and allowed to explore freely. Mice were observed walking along the edge of the cage and lowering themselves into it, and scored accordingly: 0 = Confident walk and good landing, 1 = Trips and wobbles while walking, 2 = Trips and wobbles, slips from ledge but recovers; 3 = unable to walk along ledge

Hindlimb clasping test: The mouse was grasped by the tail near its base and suspended in the air for 10 seconds. Position of hindlimbs was observed and scored as follows: 0 = Hindlimbs consistently pointing outward away from abdomen, 1 = Hindlimbs pulled in slightly towards body for more than 50% of the time suspended, 2 = Hindlimbs pointed downwards towards abdomen for more than 50% of the time suspended, 3 = Hindlimbs entirely retracted and touching the abdomen for more than 50% of the time suspended.

Gait test: The animal was placed on a flat surface with its head facing away from investigator, then observed from behind as it walked and behavior was scored as follows: 0 = Mouse moves normally, body weight supported on all limbs, abdomen not touching ground and both hindlimbs participating evenly, 1= Slight tremor observed, slightly raised pelvis or slight waddle, 2 = Severe tremor, raised pelvis or pronounced waddle, 3 = Movements disjointed, stuttering with raised pelvis and severe waddle. Mouse might not move much at all.

Kyphosis test: The mouse was placed on a flat surface and observed from the side as it walked, scored as follows: 0 = Easily able to straighten its spine as it walks, 1 = Mild kyphosis (curvature of the spine) but mostly able to straighten itself as it walks, 2 = Unable to straighten spine completely and maintains mild but persistent kyphosis, 3 = Maintains pronounced kyphosis as it walks or while it sits.

Grip strength manometry: Limb muscle strength was measured at ages P21-25, and will again be measured at P30 and P60 using a grip strength meter (Bioseb®). Grip strength from front limbs is measured in triplicate with a 1-minute break in between each test to allow the mouse to rest. For each test the mouse is held by the base of the tail and lowered onto the grid until it gripped with front paws.

### Results

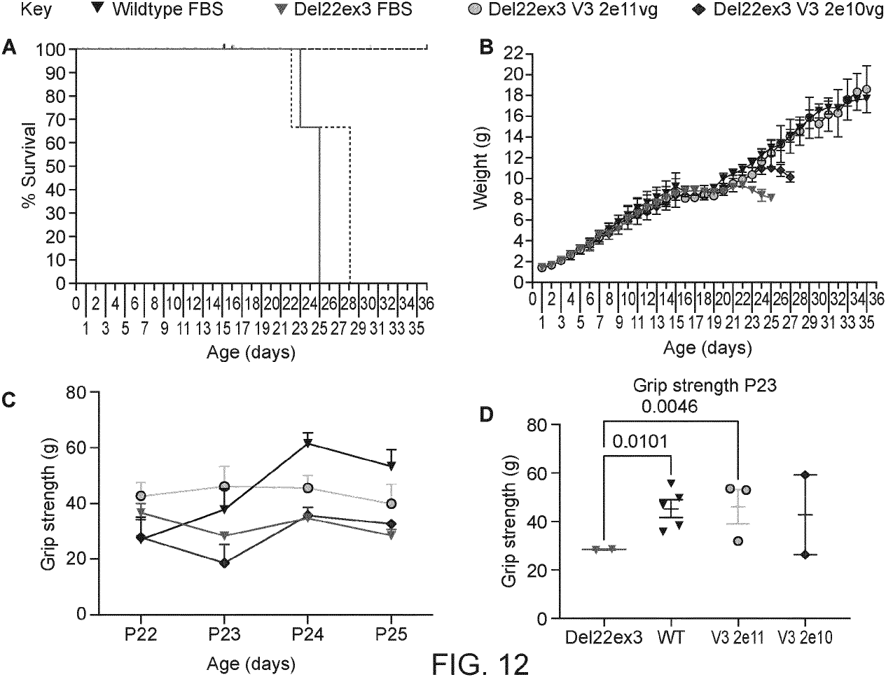

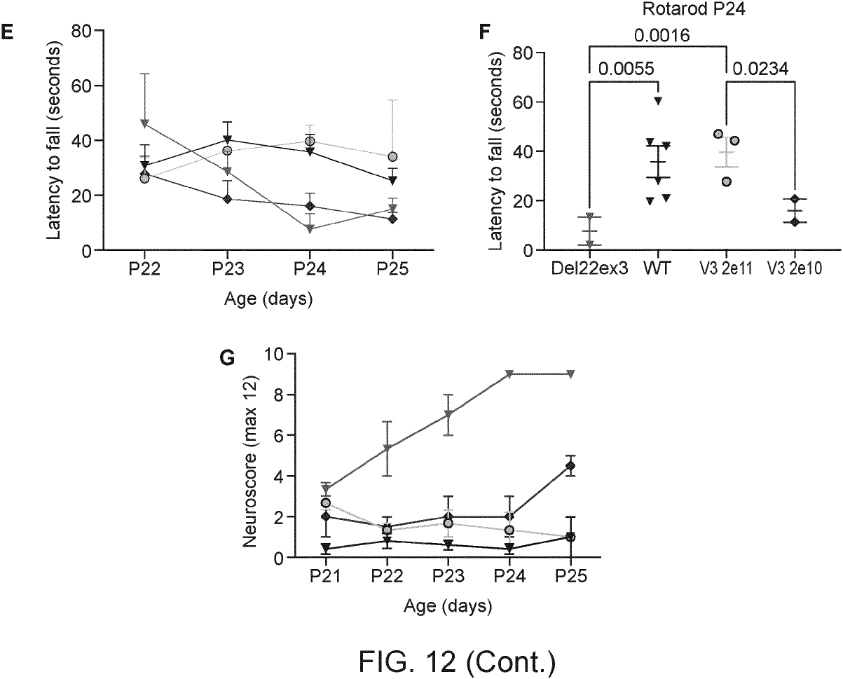

FIG. 12A Kaplan-Meier survival plot of Del22ex3 mice receiving V3 high dose (2 × 10¹¹ vg/pup n=5), Del22ex3 mice receiving V3 low dose (2 × 10¹⁰ vg/pup n=5), Del22ex3 controls receiving formulation buffer solution (n=3) and wildtype controls receiving formulation buffer solution (n=6). FIG. 12B Body weight of mice across time (Data means ± S.E.M.). FIG. 12C Motor assessment by grip strength manometry P22-25 (Data means ± S.E.M)

FIG. 12D Grip strength manometry at P23. (Data means ± S.E.M., two-way ANOVA, and Tukey’s multiple comparison). FIG. 12E Motor assessment by rotarod P22-25 (Data means ± S.E.M)

FIG. 12F Rotarod at P24 (Data means ± S.E.M., two-way ANOVA, and Tukey’s multiple comparison).

FIG. 12G Neurological scores from P21-25.

### Conclusions

This experiment demonstrates a dose-related benefit with AAV9-mediated expression of eEF1A2 in homozygous Del22ex3 mice with V3. Therapeutic efficacy is demonstrated by an increase in the age of survival up to postnatal day 36 (last time point evaluated to date), body weight gain and muscle strength and motor behavior measured by grip strength and rotarod, and with amelioration of normal deterioration in neurological score. Longer survival, up to postnatal day 36 is observed in V3 high dose treated animals compared to untreated homozygous Del22ex3 controls that survive maximally to only postnatal day 25. Length of survival is also increased compared to controls, up to postnatal day 28, in the V3 low dosage group. There is increase in body weight gain comparable to wildtype littermate controls in the V3 high dosage group. Grip strength manometry shows reduced muscle strength in untreated controls, with increasing grip strength with increasing age in wildtype animals. There is increased grip strength in V3 high dosage treated animals that is sustained from P22-25 compared to untreated controls. At postnatal day 23, V3 high dosage treated mice show significantly stronger grip strength manometry (p=0.0046), equivocal to wildtype littermates. No effect is seen with V3 low dosage. Rotarod latency to fall performance shows sustained performance in V3 high dosage between postnatal days 22-25 compared to untreated and V3 low dosage, that both show trend for decline with age. At postnatal day 24, V3 high dosage treated animals show significantly higher rotarod performance compared to untreated (p=0.0016) and are comparable to wildtype littermates. No effect is seen with V3 low dosage group. Additional evidence of benefit of AAV9-eEF1A2 can be found in the neurological scores of V3-treated animals. Neuroscores in De122.ex3 untreated controls increase with age from P21-25, consistent with neurobehavioral decline. This is not observed in V3 high dose treated animals as they are comparable to wildtype littermates between postnatal days 21-25. Persistently lower neuroscores are also observed in the V3 low dosage group compared to Del22ex3 untreated animals, at least through P24.

1. A recombinant adeno-associated virus (rAAV) virion, comprising a capsid and a vector genome, wherein the vector genome comprises a polynucleotide sequence encoding an eEF1A2 protein or a functional variant thereof, operatively linked to a promoter.

2. The rAAV virion of claim 1, wherein the promoter is a neuron-specific promoter.

3. The rAAV virion of claim 1 or claim 2, wherein the promoter is a pan-neuronal promoter.

4. The rAAV virion of any one of claims 1-3, wherein the promoter is a synapsin 1 promoter.

5. The rAAV virion of claim 4, wherein the synapsin 1 promoter is a human synapsin 1 (hSYN) promoter.

6. The rAAV virion of claim 5, wherein the hSYN promoter comprises a polynucleotide sequence that shares at least 70%, at least 80%, or at least 90% identity to SEQ ID NO: 3.

7. The rAAV virion of claim 6, wherein the hSYN promoter comprises a polynucleotide sequence that shares at least 95% or at least 99% identity to SEQ ID NO: 3.

8. The rAAV virion of claim 7, wherein the hSYN promoter comprises the polynucleotide sequence of SEQ ID NO: 3.

9. The rAAV virion of any one of claims 1-3, wherein the promoter is an eSYN promoter.

10. The rAAV virion of claim 9, wherein the eSYN promoter comprises a polynucleotide sequence that shares at least 70%, at least 80%, at least 90%, at least 95%, at least 99% or 100% identity to SEQ ID NO: 64.

11. The rAAV virion of any one of claims 1-10, wherein the polynucleotide sequence encoding the eEF1A2 protein shares at least 70%, at least 80%, or at least 90% identity to SEQ ID NO: 2.

12. The rAAV virion of claim 11, wherein the polynucleotide sequence encoding the eEF1A2 protein shares at least 95% or at least 99% identity to SEQ ID NO: 2.

13. The rAAV virion of claim 12, wherein the polynucleotide sequence encoding the eEF1A2 protein comprises the polynucleotide sequence of SEQ ID NO: 2.

14. The rAAV virion of any one of claims 1-13, wherein the eEF1A2 protein shares at least 70%, at least 80%, or at least 90% identity to SEQ ID NO: 1.

15. The rAAV virion of claim 14, wherein the eEF1A2 protein shares at least 95% or at least 99% identity to SEQ ID NO: 1.

16. The rAAV virion of claim 15, wherein the eEF1A2 protein comprises the polynucleotide sequence of SEQ ID NO: 1.

17. The rAAV virion of claim 15, wherein the promoter is a synapsin 1 promoter.

18. The rAAV virion of claim 17, wherein the synapsin 1 promoter is a human synapsin 1 (hSYN) promoter.

19. The rAAV virion of claim 18, wherein the hSYN promoter comprises a polynucleotide sequence that shares at least 95% identity to SEQ ID NO: 3.

20. The rAAV virion of any one of claims 1 or 11-15, wherein the promoter is a constitutive promoter.

21. The rAAV virion of any one of claims 1, 11-15, or 20, wherein the promoter is a CAG promoter, wherein optionally the CAG promoter shares at least 95% identity with SEQ ID NO: 14.

22. The rAAV virion of any one of claims 1, 11-15, or 20, wherein the promoter is a CMV promoter, wherein optionally the CMV promoter comprises a sequence at least 95% identical to SEQ ID NO: 16 or 17.

23. The rAAV virion of any one of claims 1-22, wherein the vector genome comprises polyadenylation (polyA) site.

24. The rAAV virion of claim 23, wherein the polyA sequence is a bGH polyadenylation site.

25. The rAAV virion of claim 24, wherein the bGH polyadenylation site shares at least 95% identity to SEQ ID NO: 53.

26. The rAAV virion of claim 23, wherein the polyA sequence is a hGH polyadenylation site.

27. The rAAV virion of claim 26, wherein the hGH polyadenylation site shares at least 95% identity to SEQ ID NO: 54.

28. The rAAV virion of any one of claims 1-27, wherein the vector genome comprises a WPRE(x) element.

29. The rAAV virion of claim 28, wherein the WPRE(x) element shares at least 95% identity to SEQ ID NO: 42.

30. The rAAV virion of claim 28, wherein the WPRE(x) element shares at least 95% identity to SEQ ID NO: 41 or SEQ ID NO: 43.

31. The rAAV virion of any one of claims 1-30, wherein the vector genome comprises a Kozak sequence.

32. The rAAV virion of claim 31, wherein the Kozak sequence is SEQ NO: 10.

33. The rAAV virion of any one of claims 1-32, wherein the vector genome comprises a 5′ untranslated region (UTR) that shares at least 95% identity to one or more of SEQ ID NOs: 32-40.

34. The rAAV virion of any one of claims 1-33, wherein the vector genome comprises a 3′ untranslated region (UTR) that shares at least 95% identity to one or more of SEQ ID NOs: 41-49.

35. The rAAV virion of any one of claims 1-34, wherein the vector genome comprises a 5′ inverted terminal repeat (ITR) having a sequence at least 95% identical to SEQ ID NO: 19 or SEQ ID NO: 20.

36. The rAAV virion of any one of claims 1-35, wherein the vector genome comprises a 3′ inverted terminal repeat (ITR) having a sequence at least 95% identical to SEQ ID NO: 21 or SEQ ID NO: 63.

37. The rAAV virion of claim 21, wherein the CAG promoter shares at least 95% identity with SEQ ID NO: 14.

38. The rAAV virion of any one of claims 1-36, wherein the capsid is an AAV9 capsid or functional variant thereof.

39. The rAAV virion of any one of claims 1-38, wherein the capsid shares at least 98%, 99%, or 100% identity to SEQ ID NO: 15.

40. The rAAV virion of any one of claims 1-39, wherein the vector genome comprises an expression cassette, the expression cassette comprising, in 5′ to 3′ order:
a. HuBA promoter, the polynucleotide sequence encoding eEF1A2 or a functional variant thereof, WPRE(x), and pAGlobin-Oc;
b. CMV promoter, TPL-eMLP enhancer, the polynucleotide sequence encoding eEF1A2 or a functional variant thereof, WPRE(r), and pAGlobin-Oc;
c. Syn promoter, the polynucleotide sequence encoding eEF1A2 or a functional variant thereof, WPRE(r), 3′UTR (globin), and pAGH-Bt.
d. CBA promoter, the polynucleotide sequence encoding eEF1A2 or a functional variant thereof, and pAGH-Bt;
e. EF1α promoter, the polynucleotide sequence encoding eEF1A2 or a functional variant thereof, and pAGlobin-Oc;
f. HuBA promoter, the polynucleotide sequence encoding eEF1A2 or a functional variant thereof, R2V17, and pAGH-Bt;
g. Syn promoter, the polynucleotide sequence encoding eEF1A2 or a functional variant thereof, WPRE(x), 3′UTR (globin), and pAGH-Hs;
h. CaMKIIa promoter, the polynucleotide sequence encoding eEF1A2 or a functional variant thereof, WPRE(r), and pAGH-Hs.
i. CMV promoter, TPL-eMLP enhancer, the polynucleotide sequence encoding eEF1A2 or a functional variant thereof, WPRE(r), and pAGH-Hs;
j. HuBA promoter, the polynucleotide sequence encoding eEF1A2 or a functional variant thereof, and pAGH-Hs.
k. CMV promoter, TPL/eMLP enhancer, the polynucleotide sequence encoding eEF1A2 or a functional variant thereof, R2V17, 3′UTR (globin), and pAGH-Bt;
1. EF1α promoter, the polynucleotide sequence encoding eEF1A2 or a functional variant thereof, WPRE(r), and pAGH-Bt;
m. Syn promoter, the polynucleotide sequence encoding eEF1A2 or a functional variant thereof, R2V17, and pAGlobin-Oc;
n. CaMKIIa promoter, the polynucleotide sequence encoding eEF1A2 or a functional variant thereof, R2V17, and pAGlobin-Oc;
o. CBA promoter, the polynucleotide sequence encoding eEF1A2 or a functional variant thereof, WPRE(x), 3′UTR (globin), and pAGH-Hs;
p. CBA promoter, the polynucleotide sequence encoding eEF1A2 or a functional variant thereof, 3′UTR (globin), and pAGlobin-Oc.
q. CaMKIIa promoter, the polynucleotide sequence encoding eEF1A2 or a functional variant thereof, R2V17, and pAGH-Bt.
r. EF1α promoter, the polynucleotide sequence encoding eEF1A2 or a functional variant thereof, R2V17, 3′UTR (globin), and pAGH-Hs.
s. CMV promoter, the polynucleotide sequence encoding eEF1A2 or a functional variant thereof, R2V17, 3′UTR (globin), and pAGH-Hs.
t. CMV promoter, the polynucleotide sequence encoding eEF1A2 or a functional variant thereof, and pAGH-Hs;
u. hSYN promoter, the polynucleotide sequence encoding eEF1A2 or a functional variant thereof, WPRE(x), and pAGH-Bt;
v. hSYN promoter, the polynucleotide sequence encoding eEF1A2 or a functional variant thereof, WPRE(x), and pAGH-Hs;
w. hSYN promoter, Kozak, the polynucleotide sequence encoding eEF1A2 or a functional variant thereof, WPRE(x), and pAGH-Hs;
x. CAG promoter, the polynucleotide sequence encoding eEF1A2 or a functional variant thereof, WPRE(x), and pAGH-Hs;
y. CAG promoter, Kozak, the polynucleotide sequence encoding eEF1A2 or a functional variant thereof, WPRE(x), and pAGH-Hs;
z. hSYN promoter, the polynucleotide sequence encoding eEF1A2 or a functional variant thereof, WPRE(x), and pAGH-Bt;
aa. hSYN promoter, the polynucleotide sequence encoding eEF1A2 or a functional variant thereof, and pAGH-Hs;
bb. hSYN promoter, Kozak, the polynucleotide sequence encoding eEF1A2 or a functional variant thereof, and pAGH-Hs;
cc. CAG promoter, the polynucleotide sequence encoding eEF1A2 or a functional variant thereof, and pAGH-Hs; or
dd. CAG promoter, Kozak, the polynucleotide sequence encoding eEF1A2 or a functional variant thereof, and pAGH-Hs.

41. The rAAV virion of any one of claim 1-40, wherein the vector genome comprises, consists essentially of, or consists of a polynucleotide sequence that shares at least 90%, 95%, 99%, or 100% identity to any one of SEQ ID NOs: 55-58 or 65-68.

42. A method of treating and/or preventing a neurological disease or disorder in a subject in need thereof, comprising administering the rAAV virion of any one of claims 1-41 to the subject.

43. The method of claim 42, wherein the subject has or is suspected of having one or more mutations in the EEFIA2 gene.

44. The method of claim 42 or claim 43, wherein the neurological disease or disorder comprises epilepsy.

45. The method of any one of claims 42-44, wherein the neurological disease or disorder comprises intellectual disability.

46. The method of any one of claims 42-45, wherein the neurological disease or disorder comprises autism.

47. The method of any one of claims 42-46, wherein the administering step comprises intracerebroventricular administration.

48. The method of any one of claims 42-47, wherein the administering step comprises intravenous administration.

49. The method of any one of claims 42-48, wherein the administering step comprises, concurrently or sequentially, both intracerebroventricular administration and intravenous administration.

50. The method of any one of claims 42-49, wherein the subject is a mammal.

51. The method of any one of claims 42-50, wherein the method prevents a decline in neurological performance in the subject compared to an untreated control subject.

52. The method of any one of claims 24-51, wherein the method improves muscle strength and/or motor skills, wherein optionally muscle strength and/or motor skills is evaluated using an inverted grid test or a rota rod test.

53. A method of expressing eEF1A2 in brain of a subject in need thereof, comprising administering the rAAV virion of any one of claims 1-41 to the subject.

54. The method of claim 53, wherein the method causes higher expression in the forebrain, wherein optionally the expression to compared to a reference vector comprising a vector genome comprising an hSYN promoter, a Kozak sequence, an eEF1A2 transgene, and/or a human globulin polyadenylation sequence (hGH).

55. The method of claim 53, wherein the method causes higher expression in the cortex, wherein optionally the expression to compared to a reference vector comprising a vector genome comprising an hSYN promoter, a Kozak sequence, an eEF1A2 transgene, and/or a human globulin polyadenylation sequence (hGH).

56. The method of any one of claims 53-55, wherein the vector genome of the rAAV virion does not comprise a Kozak sequence.

57. The method of any one of claims 53-56, wherein the vector genome of the rAAV virion does not comprise a human globulin polyadenylation sequence (hGH).

58. The method of any one of claims 53-57, wherein the vector genome of the rAAV virion comprises a bovine globulin polyadenylation sequence (bGH).

59. The method of any one of claims 53-58, wherein the subject has or is suspected of having one or more mutations in the EEF1A2 gene.

60. The method of any one of claims 53-59, wherein the subject suffers from or is at risk for a neurological disease or disorder.

61. The method of claim 60, wherein the neurological disease or disorder comprises epilepsy.

62. The method of claim 60 or claim 61, wherein the neurological disease or disorder comprises intellectual disability.

63. The method of any one of claims 60-62, wherein the neurological disease or disorder comprises autism.

64. The method of any one of claims 53-63, wherein the administering step comprises intracerebroventricular administration.

65. The method of any one of claims 53-63, wherein the administering step comprises intravenous administration.

66. The method of any one of claims 53-63, wherein the administering step comprises, concurrently or sequentially, both intracerebroventricular administration and intravenous administration.

67. The method of any one of claims 53-67, wherein the subject is a mammal.

68. The method of any one of claims 53-67, wherein the method prevents a decline in neurological performance in the subject compared to an untreated control subject.

69. The method of any one of claims 53-68, wherein the method improves muscle strength and/or motor skills, wherein optionally muscle strength and/or motor skills is evaluated using an inverted grid test or a rota rod test.

70. A pharmaceutical composition comprising the rAAV virion of any one of claims 1-41.

71. A kit comprising the rAAV virion of any one of claims 1-41 and instructions for use.


SEQUENCE LISTING (68 sequences)

SEQ ID NO: 1 - protein, 463 aa, Homo sapiens
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 2 - DNA, 1389 bp, Homo sapiens
atgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaag

SEQ ID NO: 3 - DNA, 448 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 4 - DNA, 1392 bp, Artificial Sequence
  Codon optimized eEF1A2 polynucleotide variant
atgggtaaagaaaaaacacatattaatatagtagtaatcggtcatgttgactctggaaaatctactactacaggacatttgatttataaatgtggaggaattgataaaagaacaatagaaaaatttgaaaaagaagctgctgaaatgggtaaaggtagttttaaatatgcttgggttttggataaattgaaagctgaaagagaaagaggaattacaattgatatttctttgtggaaatttgaaactacaaaatattatataacaataatagatgctcctggacatagagattttattaaaaatatgattacaggaacttctcaagcagattgtgctgttttgatagtagcagcaggagttggtgaattcgaagcaggcatttctaaaaatggacaaactagagaacatgctttgttggcttatacattgggcgtaaaacaattgattgtaggagttaataaaatggattctactgaacctgcatattctgaaaaaagatatgatgaaatagtaaaagaagtttctgcttatattaaaaaaattggttataatcctgctacagttccatttgttcctatttctggatggcatggagataatatgttggaacctagtcctaatatgccttggtttaaaggatggaaagttgaaaggaaagaaggaaatgcatcaggagtctccttgttggaagctttggatacaatcttgcctccaacaagacctacagataaacctttgagattgcctcttcaagatgtatataaaataggaggaataggaacagtgccagttggaagagtagaaacaggtatattgagacctggaatggttgtaacatttgcaccagttaatataactactgaagtaaaatctgttgaaatgcatcatgaagctttgtctgaagctcttcctggagataatgtaggatttaatgttaaaaatgtaagtgtaaaagatataagaagaggaaatgtatgtggtgatagtaaatcagatccacctcaagaagcagctcaatttacatcacaagtaataatattgaatcatcctggacaaatttctgcaggatattcaccagtaatagattgtcatacagcacatatagcttgtaaatttgctgaattgaaagaaaaaattgatagaagaagtggaaaaaaacttgaagataatcctaaatcattgaaatcaggagatgcagctattgtagaaatggtacctggaaaaccaatgtgtgtagaatctttttctcaatatccacctctcggaagatttgctgttagagatatgagacaaacagttgcagtaggagttattaaaaatgtagaaaaaaaaagcggaggtgcaggaaaggttacaaaatccgcacaaaaagctcaaaaagctggtaaataa

SEQ ID NO: 5 - DNA, 1392 bp, Artificial Sequence
  Codon optimized eEF1A2 polynucleotide variant
atgggcaaagaaaaaacacatataaacattgtcgttatcggacacgttgattctggtaaaagtacaacaaccggtcacttgatatacaaatgcgggggtatagacaaacgcactattgaaaagttcgagaaagaagctgcggagatgggcaaaggctcattcaagtacgcgtgggtactcgataagttgaaagctgaacgcgagaggggaatcaccatagacatctcactttggaaattcgagacaaccaagtattacataactattatagatgccccaggccacagggatttcattaaaaatatgataaccggcacatctcaagccgattgcgccgtactcatcgtcgccgctggtgtgggtgagttcgaggcaggtatttctaaaaatggccagacacgcgaacatgctcttctggcttatacactcggggttaaacagctcatagtaggagtgaataagatggactccactgaacccgcctattcagagaagcgctatgacgaaattgtaaaggaggtctcagcatatattaaaaaaattggctataacccagccacggtgccattcgtcccgattagtggatggcatggtgacaatatgctggaaccaagtcccaatatgccttggtttaagggttggaaagtagagcggaaagagggtaatgcttccggcgtgtcattgctggaggcgcttgacacgatactcccacccacaaggccaactgataagccactccgattgcccttgcaggacgtgtacaagattgggggaattgggactgtgcccgtcgggcgcgtggagacgggcatcctcagacctgggatggtagtcacttttgcccccgtcaacataacgactgaagttaaatcagtggaaatgcatcacgaagctttgagtgaggcgcttcccggagataacgttggatttaatgtcaaaaatgtctccgttaaagatataagaagaggaaacgtctgcggtgactcaaagtcagacccaccacaggaggctgctcaatttacgagtcaagtaataattctgaatcaccctgggcaaataagtgcgggatactctccagtcatcgattgtcacaccgcccatattgcatgtaagttcgcagaacttaaggaaaagatcgaccgaagaagcggaaaaaaattggaagataatccgaaaagtttgaaaagcggtgacgcggcgattgtagagatggtccctggcaaaccgatgtgtgtggagtctttcagtcaatatccaccactcggtcgctttgccgtgcgggatatgcgacagaccgttgctgtcggcgtaataaaaaacgtcgaaaaaaagagcggtggggctggaaaagttacaaaatccgctcaaaaggcacagaaggcgggcaagtga

SEQ ID NO: 6 - DNA, 1392 bp, Artificial Sequence
  Codon optimized eEF1A2 polynucleotide variant
atgggtaaagaaaagacccacattaacatagtagtaatcggtcatgttgactctgggaaaagcactactaccggacatttgatctataaatgtgggggcatcgacaaaagaacgatagagaagtttgagaaggaggcggcggagatgggtaaaggtagttttaagtacgcttgggttttggacaaattgaaagccgagcgcgagcgcggcattaccattgacatttctctctggaaattcgaaactacgaagtattatataacaataatagacgcccccggccatcgggactttattaaaaacatgattacaggaactagccaagcagattgtgctgtgctgatagtagcggcaggggtcggggagttcgaagcaggcatctctaaaaatggacaaactcgagagcacgccttgttggcttataccttgggcgtaaagcagctgatcgtaggagttaataaaatggattccactgaacccgcatatagcgaaaagcgatatgacgaaatagtaaaggaagtctcagcttatatcaagaaaatcggttacaatcctgcgacggttccattcgttcctatctccgggtggcacggcgataatatgcttgagcccagtcccaatatgccctggttcaaggggtggaaggttgagaggaaggaaggcaatgcatcaggcgtcagcttgttggaagctctcgacaccatcctgccgcccacgaggcccacagacaaaccgttgcgactgcctcttcaagatgtatacaaaataggcgggataggaaccgtgccggttggacgagtagagacgggtatactgcggcccggaatggtcgtgacgtttgcacccgtgaatataactactgaggtgaagagcgtcgagatgcaccatgaagcgctgagtgaagctctccctggcgataacgtagggttcaacgtgaaaaacgtaagtgtaaaggatataaggcgcggaaatgtatgtggtgacagtaaaagcgacccgccgcaagaggcggcgcaattcacatcacaggtaataatattgaatcaccccggccaaatttccgcaggctactcaccagtcatagattgccacaccgcccacatagcttgtaagttcgctgagttgaaagagaagattgatagacgaagtgggaagaaacttgaagacaatccgaagtccctgaagtccggtgacgcagcgattgtagaaatggtaccgggcaagccaatgtgtgtagagtctttcagccagtacccaccactggggcggttcgcggtgcgagacatgaggcaaacggttgcggtcggcgtcattaaaaatgtcgaaaaaaagagtggcggtgcaggtaaggtcacaaaaagcgcacaaaaggcccagaaagccggtaagtga

SEQ ID NO: 7 - DNA, 1392 bp, Artificial Sequence
  Codon optimized eEF1A2 polynucleotide variant
atgggaaaggaaaaaactcacataaacattgtcgtcatcggtcacgtagacagtggcaaatcaacgaccactggacatctcatctataagtgtggcggtattgacaaacgcactatcgagaaattcgaaaaggaggctgctgagatgggcaaaggctctttcaagtacgcatgggtcctggataagctgaaagcggagcgagagagagggatcaccatcgatatatctctgtggaaatttgaaaccaccaagtactacatcacaattattgatgccccaggtcatagggattttatcaagaacatgatcaccgggacaagccaagccgactgcgcagttctcatagtggcggctggagtaggggagtttgaagcagggatatctaagaatggacagacccgcgagcacgccttgctggcctacaccctgggagtgaagcagctcatagttggcgtcaataagatggacagcaccgaacccgcctacagtgagaagaggtatgacgagattgtgaaggaggtttctgcttacattaaaaagattggctataacccagctactgtcccattcgttccaatcagcggctggcacggtgataacatgctggagcctagtcccaacatgccgtggttcaaggggtggaaggttgaacgcaaggaggggaatgcctcaggcgtttccctgctggaggccctcgatacaatactccccccgacccggcctacagataaaccgctgcgactgcctcttcaggacgtgtataaaatcgggggaatcggcacagtgcccgtgggcagggtagagactggcatcttgcggcctggaatggtagtcacctttgccccggttaatatcacaacggaggtgaaatctgtggagatgcatcacgaagcactgagcgaggctctgcctggtgacaacgtgggatttaacgtcaaaaacgtgtcagtcaaggacatccgccgcggtaacgtttgcggagattctaagtccgatcccccccaggaggcagcccaatttacctcccaagtgatcattctgaatcacccaggccaaatttccgccgggtattcccctgtgattgactgtcacacagcacacatcgcatgcaaattcgccgaactcaaggagaaaattgatcggagaagcggtaaaaaactggaggacaacccaaagtccctcaagtctggggatgccgccatcgtggagatggtaccaggcaaacctatgtgcgtggaaagttttagccagtaccctccactgggtcgctttgctgttcgggatatgcggcagacagtagcggttggggtcataaaaaacgtcgagaaaaagagcggaggagctgggaaagttaccaaatccgcacagaaggcacaaaaagccggaaaatga

SEQ ID NO: 8 - DNA, 1392 bp, Artificial Sequence
  Codon optimized eEF1A2 polynucleotide variant
atgggcaaagagaaaacacatattaacattgttgttatcgggcacgttgatagcggcaagtccactaccactggccatctgatttacaagtgcggcggaatcgataaacgaactattgaaaagttcgaaaaagaagccgccgagatgggaaagggctcctttaaatacgcttgggtcctcgataaactcaaagcagaacgggagagaggaatcaccatcgatatatccttgtggaagttcgaaactacaaaatattacattaccatcattgatgcgcctgggcaccgcgacttcattaagaacatgattactggcacctctcaagccgactgcgcagtgttgatcgtagccgcaggcgtcggggagttcgaagctgggatcagcaagaacgggcagactagggaacacgctctgctcgcatatactcttggcgtgaaacagttgatcgttggcgtgaacaagatggattcaactgagcctgcctattctgagaaacgatacgacgagattgtgaaagaggtttcagcttacatcaagaaaattgggtataatcccgcaacagttcccttcgtgcccatctctgggtggcacggcgacaacatgctcgaaccatccccaaatatgccatggttcaagggatggaaggtggagcgcaaagaaggcaacgcctccggagtgtctctgctcgaggccctggacaccattctgcccccaacacgacccactgataagcctctgagactgccactgcaagacgtttacaaaattgggggaattggaaccgtgcctgtgggtcgggtggaaaccggaatcctcagacccggcatggtggtcaccttcgcaccagtgaatataacgacagaggtcaaatctgtggagatgcaccatgaggcattgagcgaggcactcccaggagacaacgtgggtttcaacgtgaaaaatgtctcagttaaggacatccgacgcggcaacgtgtgcggagatagcaaatctgaccccccccaggaggccgctcaattcacaagtcaggttatcatccttaatcaccctggccaaatatctgcaggctacagccccgtgatcgattgtcacacagctcatatcgcctgtaaatttgctgaactcaaagaaaagattgaccgcagatcaggaaaaaagctggaggacaaccctaaaagtctgaagtccggcgacgctgccatcgtggagatggtccctgggaaacccatgtgcgtggagtccttttctcagtacccccctctgggacgattcgccgtgcgcgacatgagacagactgtcgccgtgggcgtcattaaaaatgtggaaaaaaaatcaggaggtgcagggaaagtgacaaagagtgcccagaaagcacagaaggctggcaagtga

SEQ ID NO: 9 - DNA, 1395 bp, Artificial Sequence
  Human eEF1A2 with Kozak sequence
gccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaag

SEQ ID NO: 10 - DNA, 10 bp, Artificial Sequence
  Kozak sequence motif
gccaccatgg

SEQ ID NO: 11 - RNA, 13 nt, Artificial Sequence
  Alternative Kozak sequence motif
gccgccrccaugg

SEQ ID NO: 12 - RNA, 10 nt, Artificial Sequence
  Alternative Kozak sequence motif
gacaccaugg

SEQ ID NO: 13 - DNA, 168 bp, Adeno-associated virus
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 14 - DNA, 573 bp, Artificial Sequence
  Made in lab - CAG promoter in part Human betaherpesvirus 5
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 15 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 220 bp, Human betaherpesvirus 5
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 17 - DNA, 583 bp, Human betaherpesvirus 5
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 18 - DNA, 141 bp, Adeno-associated virus
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 19 - DNA, 168 bp, Adeno-associated virus
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 20 - DNA, 170 bp, Adeno-associated virus
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 21 - DNA, 141 bp, Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 124 bp, Artificial Sequence
  Made in Lab - vector filler sequence
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 23 - DNA, 84 bp, Artificial Sequence
  Made in Lab - vector filler sequence
cttctgaggcggaaagaaccagatcctctcttaaggtagcatcgagatttaaattagggataacagggtaatggcgcgggccgc

SEQ ID NO: 24 - DNA, 63 bp, Artificial Sequence
  Made in Lab - vector filler sequence
gttacccaggctggagtgcagtggcacatttctgctcactgcaacctcctcctccctgggttc

SEQ ID NO: 25 - DNA, 253 bp, Homo sapiens
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 26 - DNA, 281 bp, Gallus gallus
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 27 - DNA, 455 bp, Homo sapiens
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 28 - DNA, 401 bp, Homo sapiens
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 29 - DNA, 422 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 30 - DNA, 281 bp, Homo sapiens
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 31 - DNA, 293 bp, Human betaherpesvirus 5
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 32 - DNA, 953 bp, Homo sapiens
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 33 - DNA, 1068 bp, Artificial Sequence
  Made in Lab - Chicken beta-actin exon/intron plus rabbit globin intron
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 34 - DNA, 126 bp, Homo sapiens
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 35 - DNA, 121 bp, Human betaherpesvirus 5
tcagatcgcctggagaggccatccacgctgttttgacctccatagtggacaccgggaccgatccagcctccgcggccgggaacggtgcattggaacgcggattccccgtgccaagagtgac

SEQ ID NO: 36 - DNA, 512 bp, Artificial Sequence
  Made in Lab - adenovirus derived enhancer element
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 37 - DNA, 956 bp, Homo sapiens
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 38 - DNA, 939 bp, Homo sapiens
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 39 - DNA, 83 bp, Homo sapiens
tcagaagccccgggctcgtcagtcaaaccggttctctgtttgcactcggcagcacgggcaggcaagtggtccctaggttcggg

SEQ ID NO: 40 - DNA, 476 bp, Homo sapiens
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 41 - DNA, 589 bp, Artificial Sequence
  Made in Lab - mutated woodchuck hepatitis regulatory element
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 42 - DNA, 588 bp, Artificial Sequence
  Made in Lab - mutated woodchuck hepatitis regulatory element
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 43 - DNA, 755 bp, Artificial Sequence
  Made in Lab - mutated woodchuck hepatitis regulatory element
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 44 - DNA, 12 bp, Artificial Sequence
  CAAX motif
tgtgtgataatg

SEQ ID NO: 45 - DNA, 810 bp, Homo sapiens
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 46 - DNA, 726 bp, Hepatitis B virus
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 47 - DNA, 755 bp, Artificial Sequence
  Made in Lab - HepB derived enhancer element
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 48 - DNA, 94 bp, Homo sapiens
gctggagcctcggtagccgttcctcctgcccgctgggcctcccaacgggccctcctcccctccttgcaccggcccttcctggtctttgaataaa

SEQ ID NO: 49 - DNA, 596 bp, Woodchuck hepatitis virus
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 50 - DNA, 387 bp, Oryctolagus cuniculus
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 51 - DNA, 251 bp, Bos taurus
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 52 - DNA, 251 bp, Bos taurus
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 53 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 54 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 55 - DNA, 3144 bp, Artificial Sequence
  Made in Lab - vector genome construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 3035 bp, Artificial Sequence
  Made in Lab - vector genome construct
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 57 - DNA, 3263 bp, Artificial Sequence
  Made in Lab - vector genome construct
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 58 - DNA, 4299 bp, Artificial Sequence
  Made in Lab - vector genome construct
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagcgccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 59 - protein, 735 aa, Adeno-associated virus 2 [sequence omitted]

SEQ ID NO: 60 - protein, 743 aa, Artificial Sequence
  Synthetic construct - AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 7 aa, Artificial Sequence
  Peptide insert
TLAVPFK

SEQ ID NO: 62 - protein, 7 aa, Artificial Sequence
  Peptide insert
KFPVALT

SEQ ID NO: 63 - DNA, 168 bp, Adeno-associated virus; identical to SEQ ID NO: 13

SEQ ID NO: 64 - DNA, 851 bp, Artificial Sequence
  Made in Lab - eSYN promoter polynucleotide
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagg

SEQ ID NO: 65 - DNA, 3014 bp, Artificial Sequence
  Artificial Vector Genome
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 66 - DNA, 3049 bp, Artificial Sequence
  Artificial Vector Genome
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 67 - DNA, 3277 bp, Artificial Sequence
  Artificial Vector Genome
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagagtctgcggtgggcagcggaggagtcgtgtcgtgcctgagagcgcagctgtgctcctgggcaccgcgcagtccgcccccgcggctcctggccagaccacccctaggaccccctgccccaagtcgcagccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 68 - DNA, 3498 bp, Artificial Sequence
  Artificial Vector Genome
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggccaccatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc